(19) United States
(12) Patent Application Publication
(10) Pub. No.: US 2003/0157137 A1
(43) Pub. Date: Aug. 21, 2003

(54) Emulsified skin care composition containing salicylic acid, lanolin oil, and propylene glycol

(75) Inventor: Elliott Farber, North Mankato, MN (US)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Alwyn Company, Inc.

(21) Appl. No.: 10/322,811

(22) Filed: Dec. 19, 2002

Related U.S. Application Data
(60) Provisional application No. 60/341,989, filed on Dec. 19, 2001.

(51) Int. Cl. A61K 7/06; A61K 7/00

(52) U.S. Cl. 424/401; 424/74

(57) ABSTRACT

A composition comprising an oil-in-water emulsion for skin care containing salicylic acid or another hydroxy acid, and propylene glycol to prevent formation of crystals of the hydroxy acid. The composition includes, as an emulsifier, at least one nonionic polyoxyethylene ether with a HLB of about 15 to about 17; in some embodiments, another nonionic polyoxyethylene ether with a HLB of 10 to 12 is also included. The composition also includes lanolin oil and a complex comprising Vitamin A and Vitamin D₃. Other ingredients, such as a hydrophilic carbohydrate-containing polymer or a quaternary ammonium-containing polymer, can be included. Still other ingredients, such as preservatives, chelators, herbal extracts, allantoin and fragrance, can be included. Another aspect of the invention is a method of use of a composition according to the present invention in treating dermatological or systemic inflammatory conditions such as psoriasis, seborrheic dermatitis, and dandruff.

### CROSS-REFERENCE TO RELATED APPLICATIONS

This application claims priority from U.S. Provisional Patent Application No. 60/341,989, filed Dec. 19, 2001, entitled “Emulsified Skin Care Composition Containing Salicylic Acid, Lanolin Oil, and Cod Liver Oil.”

### BACKGROUND OF THE INVENTION

1. Field of the Invention

This invention is directed to an improved emulsified skin care composition containing salicylic acid and propylene glycol that is particularly useful for treatment and/or palliation of dermatological or systemic inflammatory conditions such as psoriasis, seborrheic dermatitis and dandruff.

2. Description of the Related Art

Despite recent improvements in its treatment, psoriasis remains a serious disease of great importance. The lesions of psoriasis can be painful, unsightly, and disfiguring. Patients suffering from psoriasis are reluctant to be seen in public or undertake activities where the nature of their disease becomes manifest to the population as a whole. Accordingly, the lives of such people are impacted and restricted.

The pathological mechanisms underlying psoriasis are not completely known, although much has been learned about these applications.

Psoriasis is an inherited skin disease, the causal mechanisms of which are still unclear. In general, psoriasis is characterized by excessively rapid epidermal cell turnover. The disease is believed to have a strong genetic basis. (H. Baker, “Psoriasis” in Textbook of Dermatology (A. Rook et al., eds., 4th ed., Blackwell Scientific Publications, Boston, 1986), vol. 2, pp. 1469-1532). In fact, this has been confirmed by recent findings from genetic studies of psoriatic families (G. Lomholt, “Psoriasis: Prevalence, Spontaneous Course, and Genetics” (Copenhagen, Denmark, GEC GAD, 1963); E. M. Farber et al., “Natural History of Psoriasis in 61 Twin Pairs,” Arch. Dermatol. 109:207 (1974)), which suggest that at least two genes are implicated in the manifestation of psoriasis in predisposed individuals (J. T. Elder et al., “The Genetics of Psoriasis,” Arch. Dermatol. 130:216-224 (1994)). One of these has been mapped to the short arm of the 6th chromosome (J. T. Elder et al. (1994), supra), and the other to the distal end of chromosome 17q (J. Tomfohrde et al., “Gene for Familial Psoriasis Susceptibility Mapped to the Distal End of Human Chromosome 17q,” Science 264:1141-1145 (1994)). Chromosome 6 contain genes encoding not only class I and class II antigens of the major histocompatibility complex, but also class III (tumor necrosis factor-alpha [TNFα] molecules (J. T. Elder et al. (1994), supra). The expression of TNFα and its receptors has been shown to be enhanced in psoriatic skin (M. Kristensen et al., “Localization of Tumor Necrosis Factor-Alpha (TNFα) and Its Receptors in Normal and Psoriatic Skin: Epidermal Cells Express the 55 kD but not the 75 kD TNF Receptor,” Clin. Exp. Immun. 94:354-362 (1993)), pointing to the possible role of superantigens in precipitating the disease (M. C. Y. Heng et al., “Erythroderma Associated with Mixed Lymphocyte-Endothelial Cell Interaction and Staphylococcus aureus Infection,” Br. J. Dermatol. 115:693-705 (1986); B. J. Nickoloff et al., “Activated Keratinocytes Present Bacterial-Derived Superantigens to T Lymphocytes: Relevance to Psoriasis,” J. Dermatol. Sci. 6:127-133 (1993)).

It has been suggested that increased phosphorylase kinase activity may be due to defective regulation or presence of multiple copies of sequences encoding phosphorylase kinase in the genome of the psoriatic individual. This explanation is unlikely since, of the protein moieties under consideration, only the gene sequences encoding the regulatory subunit of cAMP-dependent protein kinase II has been mapped to the 17^{th} chromosome (P. J. Barnard et al., “Mapping of the Phosphorylase Kinase Alpha Subunit Gene on the Mouse X Chromosome,” Cytogenet. & Cell Genet. 53:91-94 (1990)). On the other hand, none of the phosphorylase kinase subunits have been mapped to chromosome 17. Instead, the alpha subunit of phosphorylase kinase has been mapped to the X chromosome, where it lies between the genes encoding the X-linked zinc finger protein and phosphoglycerate kinase genes (P. J. Williams et al., “Mapping of the Gene for X-Linked Liver Glycogenosis due to Phosphorylase Kinase Deficiency to Human Chromosome Region Xp22,” Genomics 9:565-569 (1991); M. W. Kilimann, “Molecular Genetics of Phosphorylase Kinase: cDNA Cloning, Chromosomal Mapping and Isoform Structure,” J. Inher. Metab. Dis. 13:435-441 (1990)); the β subunit to chromosome 16 (T. A. Jones et al., “Localization of the Gene Encoding the Catalytic Gamma Subunit of Phosphorylase Kinase to Human Chromosome Bands 7p 12-q21,” Biochim. Biophys. Acta 1048:24-29 (1990)), the catalytic subunit to chromosome 7 (N. J. Lowe & H. B. Ridgeway, “Generalized Pustular Psoriasis Precipitated by Lithium,” N. Eng. J. Med. 114:1788-1789 (1978)), and the calmodulin (δ) subunit to chromosome 10.

The psoriatic plaque is characterized by hyperproliferative epidermal kinetics (E. J. Van Scott & T. M. Ekel, “Kinetics of Hyperplasia in Psoriasis,” Arch. Dermatol. 88:373-381 (1963); R. Marks, “Epidermal Activity in the Involved and Uninvolved Skin of Patients with Psoriasis,” Br. J. Dermatol. 98:399-404 (1978)), increased polyamine-dependent (N. J. Lowe, “Cutaneous Polyamines in Psoriasis,” Br. J. Dermatol. 107:21-25 (1982)) cell cycling with an increased proliferative pool (G. D. Weinstein & J. L. McCollough, “Cytokinetics in Diseases of Epidermal Hyperplasia,” Annu. Rev. Med. 24:345-352 (1973); S. Gelfant; “The Cell Cycle In Psoriasis,” Br. J. Dermatol. 95:577 (1976); G. L. Grove, “Epidermal Cell Kinetics in Psoriasis,” Int. J. Dermatol. 18:111-122 (1979)), followed by increased DNA synthesis (L. Rowe et al., “Mitoses in Normal and Psoriatic Epidermis,” Br. J. Dermatol. 98:293-299 (1978)) and mitoses (J. G. Chafouleas et al., “Changes in Calmodulin and Its mRNA Accompany Reentry of Quiescent (GO) Cells Into the Cell Cycle,” Cell 36:73-81 (1984)). Polyamine-induced cell cycling involves calcium-dependent (C. Cochet & E. M. Chamber, “Polyamine-Mediated Protein Phosphorylations: A Possible Target of Intracellular Polyamine Reactions,” Mol. Cell. Endocrinol. 30:247-266 (1983)) protein phosphorylation (W. L. Combest, “Polyamines Differentially Inhibit Cyclic-AMP Dependent Protein Kinase-Mediated Phosphorylation in the Brain of a Tobacco Hornworm, Manduca sexta,” J. Neurochem. 119:1581-1591 (1988)), and ATP generation, which is achieved not directly but through an unknown substrate (W. L. Combest, 1988, supra).

Cell cycling in psoriatic epidermis has been shown additionally to be modulated by epidermal growth factor (EGF) (J. T. Elder et al., “Overexpression of TGF-α in Psoriatic Epidermis,” Science 243: 811-814 (1989); L. B. Nanney et al., “Altered [1251]-Epidermal Growth Factor Binding and Receptor Distribution in Psoriasis,” J. Invest. Dermatol. 86: 260-266 (1986)).

Another hallmark of psoriasis is blood vessel abnormalities, which precede the development of overt histological psoriasis. High endothelial venules (HEVs) exist in psoriatic skin, and tannic acid staining material is present in the intercellular spaces between adjacent endothelial cells of the HEVs in psoriatic skin. The HEVs appear to be recognized by T8 (CD8) (cytotoxic/suppressor) lymphocytes, as the presence of HEVs was found to be related to the presence of T8 (CD8) lymphocytes in the epidermis. The tannic acid staining material may serve as a marker for HEVs recognized by the T8 (CD8) lymphocyte subset. The prior existence of HEVs in uninvolved psoriatic skin could account for the rapid egress of T8 (CD8) lymphocytes from the vasculature to the epidermis in response to trauma (M. C. Y. Heng et al., “High Endothelial Venules in Involved and Uninvolved Psoriatic Skin: Recognition by Homing Receptors on Cytotoxic T Lymphocytes,” Br. J. Dermatol. 118: 315-326 (1988).

Psoriatic lesions can be induced by trauma in psoriatic individuals. Among the earliest changes noted in events leading to the formation of a psoriatic plaque induced by tape-stripping was increased mobility of the epidermal Langerhans cells across the basement membrane, evidence of Langerhans cell-lymphocyte interaction, and increased Langerhans cell activity or cytotoxicity. Collections of epidermal lymphocytes showing the features of blastoid transformation while in contact with processes from activated Langerhans cells suggest the involvement of 1 a antigens in this process. These findings are consistent with an increased immune responsiveness to trauma, controlled by genes located at the HLA-D locus of the major histocompatibility complex, and mediated by enhanced cellular interactions (M. C. Y. Heng et al., “The Sequence of Events in Psoriatic Plaque Formation After Tape-Stripping,” Br. J. Dermatol. 112: 517-532 (1985)).

There is some evidence that enhanced terminal differentiation of psoriatic keratinocytes, as shown by the increased expression of L-fucose specific binding sites on psoriatic keratinocytes (H. Roelfzema et al., “Studies on the Plasma Membrane of Normal and Psoriatic Keratinocytes. IV. Characterization of Glycoconjugates,” Br. J. Dermatol. 105:509-516 (1981); M. C. Y. Heng et al., “Expression of the L-Fucose Moiety on Epidermal Keratinocytes in Psoriasis Induced by the Koebner Phenomenon,” Br. J. Dermatol. 126:575-581 (1992); J. D. Zieski & I. A. Bernstein, “Modification of Cell Surface Glycoprotein: Addition of Fucosyl Residues During Epidermal Differentiation,” J. Cell. Biol. 95:626- 631 (1982)), may be equally important in the psoriatic process. Enhanced terminal differentiation has been shown to be an essential feature of the positive Koebner phenomenon, and L-fucose expression on epidermal keratinocytes is observed on keratinocytes undergoing terminal differentiation, such as in the Koebner phenomenon.

Biochemically, the process of terminal differentiation consists of a series of calcium-dependent reactions, all requiring energy and triggered by an influx of calcium into the keratinocyte (B. A. Dale et al., “Identification of Filaggrin in Cultured Mouse Keratinocytes and its Regulation by Calcium,” J. Invest. Dermatol. 81:90s-95s (1983); H. Hennings et al., “Calcium Regulation of Growth and Differentiation of Mouse Epidermal Cells in Culture,” Cell 19:245-254 (1980); S. T. Boyce & R. G. Ham, “Calcium Regulated Differentiation of Normal Human Keratinocytes in Chemically Defined Clonal Culture and Serum Free Serial Culture,” J. Invest. Dermatol. 81:33s-40s (1983); C. K. Menon & P. M. Elias, “Ionic Calcium Reservoirs in Mammalian Epidermis: Ultrastructural Localization With Ion-Capture Cytochemistry,” J. Invest. Dermatol. 84:508-512 (1985); P. Cohen, “The Role of cAMP-Dependent Protein Kinase in the Regulation of Glycogen Metabolism in Mammalian Skeletal Muscle,” Curr. Top. Cell. Regulat. 14:117-196 (1978)).

Among the ingredients that have been proposed for treatment of psoriasis is salicylic acid. The use of salicylic acid as a monograph approved active ingredient for treatment of psoriasis, seborrheic dermatitis, acne, and dandruff has been documented in the Federal Register, 56 (No. 233), 63554-63569 (1991).

Emulsions prepared in the usual cream and lotion bases with salicylic acid and ointments of salicylic acid in petrolatum have been observed to form large crystals or shards of salicylic acid. This crystalline formation is due to the low solubility of salicylic acid in these media and the resulting precipitation and crystal formation of salicylic acid with time. These shards of salicylic acid can be felt as the cream or ointment is spread out over the skin. Although these shards are brittle, the salicylic acid crystals are potential mechanical abrasives and irritants to the skin.

Salicylic acid can be present in such compositions at a weight/weight concentration of from about 1.8% to about 3.0%. The acidity of such systems can range from a pH of 2.5 to a pH of 4.0. Such strongly acidic conditions tend to hydrolyze nonionic esters which are frequently used as emulsifiers. This high degree of acidity also tends to hydrolyze certain ionic emulsifiers such as sodium or ammonium lauryl sulfate, making these unsuitable for the production of stable oil-in-water emulsions.

Salicylic acid is stable in alcohols and glycols. However, the formation of a stable emulsion is extremely difficult at high concentrations of alcohol or glycol. The low pH of approximately 3.0 produced by using 1.8% to 3.0% by weight of salicylic acid also contributes to emulsion instability. The concentration of 1.8% to 3.0% by weight of salicylic acid is required by the FDA for the manufacture of OTC products to treat psoriasis while only 0.50% to 2.0% by weight is necessary for an acne medicated treatment.

Accordingly, there is a need for an emulsified composition containing salicylic acid and other ingredients, that is stable at relatively low pH values and can be stored under such conditions without developing crystals of salicylic acid. There is further a need for improved methods of treatment of diseases such as psoriasis using such emulsions.

### SUMMARY OF THE INVENTION

One aspect of the present invention is an improved emulsified skin care composition comprising an oil-in-water emulsion with improved stability that contains salicylic acid. The emulsion has a relatively low pH.

A skin care composition according to a first embodiment of the present invention comprises an oil-in-water emulsion comprising:

(1) water;

(2) salicylic acid in an effective quantity;

(3) lanolin oil in an effective quantity;

(4) a complex comprising Vitamin A and Vitamin D₃ in an effective quantity;

(5) an emulsifier component in a quantity sufficient to effectively emulsify oil and water phases, the emulsifier component comprising a nonionic ether surfactant with a HLB of about 12 to about 18; and

(6) a solvent component in an effective quantity.

The pH of the composition is from about 2.0 to about 4.0.

A skin care composition according to a second embodiment of this aspect of the invention comprises an oil-in-water emulsion comprising:

(1) water;

(2) salicylic acid in an effective quantity;

(3) lanolin oil in an effective quantity;

(4) a complex comprising Vitamin A and Vitamin D₃ in an effective quantity;

(5) an emulsifier component in a quantity sufficient to effectively emulsify oil and water phases, the emulsifier component comprising:

(a) a first nonionic polyoxyethylene ether with a HLB of about 15 to about 17; and

(b) a second nonionic polyoxyethylene ether with a HLB of about 10 to about 12;

(6) a glycol in a quantity sufficient to prevent formation of salicylic acid crystals; and

(7) optionally, allantoin in an effective quantity.

A skin care composition according to a third embodiment of this aspect of the invention comprises an oil-in-water emulsion comprising:

(1) water;

(2) salicylic acid in an effective quantity;

(3) lanolin oil in an effective quantity;

(4) a complex comprising Vitamin A and Vitamin D₃ in an effective quantity;

(5) an emulsifier component in a quantity sufficient to effectively emulsify oil and water phases, the emulsifier component comprising:

(a) a first nonionic polyoxyethlene ether with a HLB of about 15 to about 17; and

(b) a second nonionic polyoxyethylene ether with a HLB of about 10 to about 12;

(6) at least one glycol in a quantity sufficient to prevent formation of salicylic acid crystals;

(7) a hydrophilic carbohydrate-containing polymer in an effective quantity; and

(8) optionally, allantoin in an effective quantity.

A skin care composition according to a fourth embodiment of this aspect of the invention comprises an oil-in-water emulsion comprising:

(1) water;

(2) salicylic acid in an effective quantity;

(3) lanolin oil in an effective quantity;

(4) a complex comprising Vitamin A and Vitamin D₃ in an effective quantity;

(5) an emulsifier component in a quantity sufficient to effectively emulsify oil and water phases, the emulsifier component comprising:

(a) a first nonionic polyoxyethylene ether with a HLB of about 15 to about 17; and

(b) a second nonionic polyoxyethylene ether with a HLB of about 10 to about 12;

(6) at least one glycol in a quantity sufficient to prevent formation of salicylic acid crystals;

(7) a quaternary ammonium-containing polymer in an effective quantity; and

(8) optionally, allantoin in an effective quantity.

The nonionic ether surfactant may be selected from the group consisting of ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-19, ceteareth-20, ceteareth-21, ceteareth-22, ceteareth-23, ceteareth-24, ceteareth-25, ceteareth-26, ceteareth-27, ceteareth-28, ceteareth-29 and ceteareth-30. Preferably, the nonionic polyoxyethylene ether with a HLB of about 15 to about 17, designated the first nonionic polyoxyethylene ether when two are used, is ceteareth-25. Preferably, the nonionic polyoxyethylene ether with a HLB of about 10 to about 12, designated the second nonionic polyoxyethylene ether when two are used, is ceteareth-6 with stearyl alcohol.

Typically, when present, the hydrophilic carbohydrate-containing polymer is selected from the group consisting of hydroxypropyl methylcellulose, hydroxyethyl ethylcellulose, methylcellulose, ethylcellulose, acacia gum, gum tragacanth, locust bean gum, guar gum, gum arabic, xanthan gum, and agarose. Preferably, the hydrophilic carbohydrate-containing polymer is hydroxypropyl methylcellulose.

Typically, the at least one glycol is selected from the group consisting of propylene glycol, ethoxydiglycol, and a mixture of propylene glycol and ethoxydiglycol.

Typically, the ammonium-containing polymer, when present, is polyquaternium-10.

Typically, the composition further comprises dimethicone.

Optionally, the composition further comprises at least one plant or herbal extract, which may be selected from the group consisting of arnica extract, St. John's wort extract, chamomile extract, and witch hazel extract.

Typically, the composition further comprises a solvent component where propylene glycol or another glycol is not already specified as an ingredient. Preferably, the solvent component comprises at least one solvent selected from the group consisting of propylene glycol, ethylene glycol, 1,3-butylene glycol, sorbitol, and glycerol. More preferably, the solvent component comprises propylene glycol.

Typically, the composition further comprises a long-chain fatty acid ester of glycerol selected from the group consisting of glyceryl stearate, glyceryl palmitate, and glyceryl arachidate. Preferably, the long-chain fatty acid ester of glycerol is glyeryl stearate.

Typically, the composition further comprises a lipid-soluble long-chain alcohol component. Preferably, the lipid-soluble long-chain alcohol component comprises at least one long-chain lipid-soluble alcohol selected from the group consisting of stearyl alcohol and cetyl alcohol. More preferably, the lipid-soluble long-chain alcohol component comprises stearyl alcohol and cetyl alcohol.

Typically, the composition further comprises caprylic/capric triglycerides.

Typically, the composition further comprises a preservative component. Preferably, the preservative component comprises at least one preservative selected from the group consisting of propylparaben, methylparaben, and 2-methyl-4-isothiazolin-3-one. More preferably, the preservative component comprises propylparaben, methylparaben, and 2-methyl-4-isothiazolin-3-one.

Typically, the composition further comprises an antioxidant component. Preferably, the antioxidant component comprises at least one antioxidant selected from the group consisting of butylated hydroxytoluene, butylated hydroxyanisole, and a complex comprising propyl gallate, propylene glycol, and citric acid. More preferably, the antioxidant component comprises butylated hydroxytoluene.

Typically, the composition further comprises a chelator component. Preferably, the chelator component is tetrasodium ethylenediaminetetraacetic acid.

Instead or in addition to salicylic acid, the composition can include another hydroxy acid. The hydroxy acid can be selected from the group consisting of α-hydroxy acids, β-hydroxy acids, and multi-hydroxy acids.

If the hydroxy acid is an α-hydroxy acid, it can be selected from the group consisting of glycolic acid, lactic acid, methyllactic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, α-hydroxylauric acid, α-hydroxymyristic acid, α-hydroxypalmitic acid, α-hydroxystearic acid, α-hydroxyarachidic acid, mandelic acid, benzylic acid, phenyllactic acid, atrolactic acid, malic acid, tartronic acid, and derivatives thereof.

Alternatively, if the hydroxy acid is an α-hydroxy acid, it can be an α-hydroxy amino acid selected from the group consisting of serine, threonine, and analogues and derivatives thereof.

If the hydroxy acid is a β-hydroxy acid, it can be a derivative of salicylic acid.

If the hydroxy acid is a multi-hydroxy acid, it can be selected from the group consisting of 4-hydroxymandelic acid, 3-hydroxy-4-methoxymandelic acid, 3-(2′-hydroxyphenyl) lactic acid, 3-(4′-hydroxyphenyl) lactic acid, 3,4-dihydroxymandelic acid, glyceric acid, erythronic acid, threonic acid, ribonic acid, arabinoic acid, xylonic acid, lyxonic acid, allonic acid, altronic acid, gluconic acid, mannoic acid, gulonic acid, idonic acid, galactonic acid, talonic acid, isomers of 2,3,4,5,6,7-hexahydroxyheptanoic acid, tartaric acid, citric acid, isomers of 2,3,4,5-tetrahydroxyhexane-1,6-dioic acid, and derivatives thereof.

Another aspect of the present invention is an encapsulated skin care composition. In general, an encapsulated skin care composition according to the present invention comprises:

(1) a shell comprising agar or gelatin;

(2) a solution of salicylic acid dissolved in at least one glycol, the solution being encapsulated in the shell; and

(3) a carrier for the shell, the carrier being in the form of a cream, gel, or ointment, the shell being broken to release the solution of salicylic acid when the composition is applied to the skin of a user.

Yet another aspect of the present invention is a method for treating dermatological or systemic inflammatory conditions such as psoriasis, seborrheic dermatitis, and dandruff. In general, a method for treating psoriasis according to the present invention comprises applying a composition according to the present invention to the skin of a patient with psoriasis in a therapeutically effective quantity.

The method can further comprise administering at least one additional topically or systemically administered agent for the treatment of psoriasis. The at least one additional topically or systemically administered agent for the treatment of psoriasis can be selected from the group consisting of calcipotriene, anthralin, psoralen, 8-methoxypsoralen, 5-methoxypsoralen, 4,5,8-trimethylpsoralen, coal tar, antihistamines, aminopterin, methotrexate, azathioprine, hydroxyurea, 6-thioguanine, cyclosporine, sulfasalazine, etretinate, isotretinoin, and colchicine.

### BRIEF DESCRIPTION OF THE DRAWINGS

FIG. 1 shows the occurrence of salicylic acid crystals (shards) after 5 weeks of aging in cream containing 7% propylene glycol;

FIG. 2 shows the occurrence of salicylic acid shards in a commercial ointment known as “Skin Plaque;” and

FIG. 3 shows the absence of salicylic acid shards after 4 months of aging in a cream containing 20% of propylene glycol.

### DETAILED DESCRIPTION OF THE PREFERRED EMBODIMENTS

### I. EMULSIFIED COMPOSITIONS CONTAINING SALICYLIC ACID

One aspect of the present invention is emulsified compositions containing salicylic acid. The composition also contains other ingredients and can optionally contain allantoin.

The invention provides for emulsified compositions containing salicylic acid and other ingredients, such as a hydrophilic carbohydrate-containing polymer or a quaternary ammonium-containing polymer. The emulsified compositions are oil-in-water emulsions. The compositions have improved stability under acidic conditions by using nonionic ether surfactants in place of nonionic ester surfactants. Theoretically, an ether bond is more stable in an acidic medium than are the chemical bonds in an ester group. Accordingly, improved emulsified compositions containing salicylic acid and a hydrophilic carbohydrate containing polymer can be prepared according to the present invention. These compositions can contain other ingredients.

The invention further provides for the inclusion of high proportions of glycols such as propylene glycol or ethoxydiglycol in the emulsion can prevent salicylic acid shards or crystals from forming. The addition of glycols may also aid in penetrating the scales that typically cover the skin of patients with psoriasis. Further, the glycols may also act as a type of preservative to prevent bacterial contamination.

A. Composition Using Single Nonionic Ether Surfactant

One embodiment of a composition according to the present invention is a composition using a single nonionic ether surfactant as an emulsifier.

In general, such a composition comprises:

(1) water;

(2) salicylic acid in an effective quantity;

(3) lanolin oil in an effective quantity;

(4) a complex comprising Vitamin A and Vitamin D₃ in an effective quantity;

(5) an emulsifier component in a quantity sufficient to effectively emulsify oil and water phases, the emulsifier component comprising a nonionic ether surfactant with a HLB of about 12 to about 18; and

(6) a solvent component in an effective quantity.

The pH of the composition is from about 2.0 to about 4.0. Preferably, the solvent component comprises at least one solvent selected from the group consisting of propylene glycol, ethylene glycol, 1,3-butylene glycol, sorbitol, and glycerol. More preferably, the solvent component comprises propylene glycol.

As used herein, the term “an effective quantity” as applied to the concentrations of salicylic acid, lanolin oil, the hydrophilic carbohydrate-containing polymer, a complex comprising Vitamin A and Vitamin D₃, and allantoin, if present, is defined as a quantity sufficient to produce an anti-inflammatory or an anti-irritation effect when applied to the skin of a user or to contribute to an anti-inflammatory or an anti-irritation effect.

The abbreviation “HLB” stands for “hydrophilic-lipophilic balance” and is a measure of the relative hydrophilicity and lipophilicity of an amphipathic molecule such as a surfactant. The higher the HLB, the greater the preference of the amphipathic molecule for water over a lipid or oil-containing environment.

Preferably, the nonionic ether surfactant is ceteareth-25. A suitable formulation of ceteareth-25 is marketed as “Cremophor A25.”

Preferred concentration ranges for these ingredients are discussed below. As indicated above, the use of allantoin is optional. Concentration ranges for allantoin, when present, are also given below.

Optionally, compositions according to this embodiment of the present invention can comprise additional ingredients.

For example, the composition typically further comprises dimethicone.

Typically, the composition further comprises a long-chain fatty acid ester of glycerol selected from the group consisting of glyceryl stearate, glyceryl palmitate, and glyceryl arachidate. Preferably, the long-chain fatty acid ester of glycerol is glyceryl stearate.

Typically, the composition further comprises a lipid-soluble long-chain alcohol component. Preferably, the lipid-soluble long-chain alcohol component comprises at least one long-chain lipid-soluble alcohol selected from the group consisting of stearyl alcohol and cetyl alcohol. More preferably, the lipid-soluble long-chain alcohol comprises stearyl alcohol and cetyl alcohol.

Typically, the composition further comprises caprylic/capric triglycerides. A suitable preparation of caprylic/capric triglycerides is marketed under the name of “Liponate GC.”

Typically, the composition further comprises a preservative component. Preferably, the preservative component comprises at least one preservative selected from the group consisting of propylparaben, methylparaben, and 2-methyl-4-isothiazolin-3-one. More preferably, the preservative component comprises propylparaben, methylparaben, and 2-methyl-4-isothiazolin-3-one. A suitable preparation of 2-methyl-4-isothiazolin-3-one is marketed by Rohm & Haas as either Neolone 5000 or Neolone 950. The 2-methyl-4-isothiazolin-3-one can also be replaced with a complex comprising methylchlorolisothiazolinone and 2-methyl-4-isothiazolin-3-one, marketed by Rohm & Haas as Kathon.

Typically, the composition further comprises an antioxidant component. Preferably, the antioxidant component comprises at least one antioxidant selected from the group consisting of butylated hydroxytoluene, butylated hydroxyanisole, and a complex comprising propyl gallate, propylene glycol, and citric acid. Most preferably, the antioxidant component comprises butylated hydroxytoluene.

Typically, the composition further comprises a chelator component. A preferred chelator component is tetrasodium ethylenediaminetetraacetic acid (tetrasodium EDTA).

Typically, the composition further comprises fragrance. The use of fragrance is well known in the cosmetic art and in the art of over-the-counter skin preparations, and need not be detailed further here; many suitable fragrances are known. In some cases, fragrance can be omitted, particularly if the composition is formulated by use for persons who are allergic to fragrance or who have particularly sensitive skin. The function and activity of compositions according to the present invention are not affected by the presence or absence of fragrance.

Optionally, the composition may further comprise at least one plant or herbal extract.

The following gives percentages for the ingredients, including the optional ingredients. These percentages, and all other percentages for ingredients in other embodiments, are given as weight percentages unless otherwise specified.

Water can comprise from about 40.0% to about 80.0% of this embodiment of the composition. Preferably, water comprises from about 45.0% to about 60.0% of this embodiment of the composition.

Salicylic acid can comprise from about 1.8% to about 3.0% of this embodiment of the composition. Preferably, salicylic acid comprises about 2.0% to about 2.8% of this embodiment of the composition.

Lanolin oil can comprise from about 2.0% to about 20.0% of this embodiment of the composition. Preferably, lanolin oil comprises from about 5.0% to about 12.0% of this embodiment of the composition.

A complex comprising Vitamin A and Vitamin D₃ can comprise from about 0.005% to about 0.50% of this embodiment of the composition. Preferably, a complex comprising Vitamin A and Vitamin D₃ comprises from about 0.005% to about 0.20% of this embodiment of the composition.

Ceteareth-25 can comprise from about 1.0% to about 6.0% of this embodiment of the composition. Preferably, ceteareth-25 comprises from about 1.5% to about 3.5% of this embodiment of the composition.

Propylene glycol can comprise from about 2.0% to about 35.0% of this embodiment of the composition. Preferably, propylene glycol comprises about 20% of this embodiment of the composition.

Tetrasodium ethylenediaminetetraacetic acid can comprise from about 0.05% to about 0.50% of this embodiment of the composition. Preferably, tetrasodium ethylenediaminetetraacetic acid comprises from about 0.10% to about 0.30% of this embodiment of the composition.

Caprylic/capric triglycerides can comprise from about 0.5% to about 5.0% of this embodiment of the composition. Preferably, caprylic/capric triglycerides comprise from about 1.0% to about 3.0% of this embodiment of the composition.

Dimethicone can comprise from about 0.5% to about 5.0% of this embodiment of the composition. Preferably, dimethicone comprises from about 1.0% to about 2.0% of this embodiment of the composition.

Cetyl alcohol can comprise from about 2.0% to about 12.0% of this embodiment of the composition. Preferably, cetyl alcohol comprises from about 3.0% to about 6.0% of this embodiment of the composition.

Stearyl alcohol can comprise from about 1.0% to about 10.0% of this embodiment of the composition. Preferably, stearyl alcohol comprises from about 2.0% to about 4.0% of this embodiment of the composition.

Glyceryl stearate can comprise from about 1.0% to about 5.0% of this embodiment of the composition. Preferably, glyceryl stearate comprises from about 1.5% to about 3.5% of this embodiment of the composition.

Butylated hydroxytoluene can comprise from about 0.1% to about 1.0% of this embodiment of the composition. Preferably, butylated hydroxytoluene comprises from about 0.4% to about 0.6% of this embodiment of the composition.

Propylparaben can comprise from about 0.10% to about 0.50% of this embodiment of the composition. Preferably, propylparaben comprises from about 0.20% to about 0.40% of this embodiment of the composition.

Methylparaben can comprise from about 0.10% to about 0.50% of this embodiment of the composition. Preferably, methylparaben comprises from about 0.20% to about 0.40% of this embodiment of the composition.

The preservative 2-methyl-4-isothiazolin-3-one can comprise from about 0.005% to about 0.08% of this embodiment of the composition. Preferably, 2-methyl-4-isothiazolin-3one comprises from about 0.02% to about 0.04% of this embodiment of the composition.

At least one plant or herbal extract, if present, can comprise from about 0.025% to about 2.50% of this embodiment of the composition.

Fragrance can comprise from about 0.10% to about 0.40% of this embodiment of the composition. Preferably, fragrance comprises from about 0.20% to about 0.40% of this embodiment of the composition. As indicated above, fragrance can be omitted.

Allantoin, if present, can comprise from about 0.01% to about 1.50% of this embodiment of the composition.

A composition according to this embodiment of the invention comprises:

(1) water;

(2) salicylic acid in an effective quantity;

(3) lanolin oil in an effective quantity;

(4) a complex comprising Vitamin A and Vitamin D₃ in an effective quantity;

(5) ceteareth-25 in a quantity sufficient to effectively emulsify oil and water phases;

(6) propylene glycol;

(7) tetrasodium ethylenediaminetetraacetic acid;

(8) caprylic/capric triglycerides;

(9) dimethicone;

(10) cetyl alcohol;

(11) stearyl alcohol;

(12) glyceryl stearate;

(13) butylated hydroxytoluene;

(14) propylparaben;

(15) methylparaben;

(16) 2-methyl-4-isothiazolin-3-one;

(17) fragrance; and

(18) optionally, allantoin in an effective quantity.

A composition according to this embodiment of the invention comprises:

(1) from about 40.0% to about 80.0% of water;

(2) from about 1.8% to about 3.0% of salicylic acid;

(3) from about 2.0% to about 20.0% of lanolin oil;

(4) from about 0.005% to about 0.30% of a complex comprising Vitamin A and Vitamin D₃;

(5) from about 1.0% to about 6.0% of ceteareth-25;

(6) from about 10.0% to about 35.0% of propylene glycol;

(7) from about 0.05% to about 0.50% of tetrasodium ethylenediaminetetraacetic acid;

(8) from about 0.5% to about 5.0% of caprylic/capric triglycerides;

(9) from about 0.3% to about 3.0% of dimethicone;

(10) from about 2.0% to about 12.0% of cetyl alcohol;

(11) from about 1.0% to about 10.0% stearyl alcohol;

(12) from about 1.0% to about 5.0% of glyceryl stearate;

(13) from about 0.1 % to about 1.0% of butylated hydroxytoluene;

(14) from about 0.10% to about 0.50% of propylparaben;

(15) from about 0.10% to about 0.50% of methylparaben;

(16) from about 0.01 % to about 0.05% of 2-methyl-4-isothiazolin-3-one; and

(17) from about 0.10% to about 0.40% of fragrance.

A composition according to this embodiment of the invention comprises:

(a) from about 45.0% to about 60.0% of water;

(b) from about 2.0% to about 2.8% of salicylic acid;

(c) from about 5.0% to about 12.0% of lanolin oil;

(d) from about 0.01% to about 0.05% of a complex comprising Vitamin A and Vitamin D₃;

(e) from about 1.5% to about 3.5% of ceteareth-25;

(f) from about 15.0% to about 22.0% of propylene glycol;

(g) from about 0.10% to about 0.30% of tetrasodium ethylenediaminetetraacetic acid;

(h) from about 1.0% to about 3.0% of caprylic/capric triglycerides;

(i) from about 1.0% to about 2.0% of dimethicone;

(j) from about 3.0% to about 6.0% of cetyl alcohol;

(k) from about 2.0% to about 4.0% stearyl alcohol;

(l) from about 1.5% to about 3.5% of glyceryl stearate;

(m) from about 0.4% to about 0.6% of butylated hydroxytoluene;

(n) from about 0.20% to about 0.40% of propylparaben;

(o) from about 0.20% to about 0.40% of methylparaben;

(p) from about 0.02% to about 0.04% of 2-methyl-4-isothiazolin-3-one; and

(q) from about 0.20% to about 0.40% of fragrance.

As an alternative to salicylic acid or in addition to salicylic acid, the composition can comprise another hydroxy acid. The use of hydroxy acids is well known in the cosmetic art and in the art of over-the-counter skin preparations. Such hydroxy acids can include, but are not limited to, α-hydroxy acids, β-hydroxy acids, and multi-hydroxy acids.

The hydroxy acid can be an α-hydroxy acid. The α-hydroxy acid can be selected from the group consisting of glycolic acid, lactic acid, methyllactic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, α-hydroxylauric acid, α-hydroxymyristic acid, α-hydroxypalmitic acid, α-hydroxystearic acid, α-hydroxyarachidic acid, mandelic acid, benzylic acid, phenyllactic acid, atrolactic acid, malic acid, tartronic acid, and derivatives thereof.

In another alternative, the α-hydroxy acid can be a α-hydroxy amino acid. The α-hydroxy amino acid can be selected from the group consisting of serine, threonine, and analogues and derivatives thereof.

Alternatively, the hydroxy acid can be a β-hydroxy acid. The β-hydroxy acid can be a derivative of salicylic acid.

In another alternative, the hydroxy acid can be a multi-hydroxy acid. The multi-hydroxy acid can be selected from the group consisting of 4-hydroxymandelic acid, 3-hydroxy-4-methoxymandelic acid, 3-(2′-hydroxyphenyl) lactic acid, 3-(4′-hydroxyphenyl) lactic acid, 3,4-dihydroxymandelic acid, glyceric acid, erythronic acid, threonic acid, ribonic acid, arabinoic acid, xylonic acid, lyxonic acid, allonic acid, altronic acid, gluconic acid, mannoic acid, gulonic acid, idonic acid, galactonic acid, talonic acid, isomers of 2,3,4,5,6,7-hexahydroxyheptanoic acid, tartaric acid, citric acid, isomers of 2,3,4,5-tetrahydroxyhexane-1,6-dioic acid, and derivatives thereof.

B. Composition Using Two Emulsifiers and Glycol

Another embodiment of the present invention is a composition that uses two emulsifiers, a first nonionic polyoxyethylene ether with a HLB of about 15 to about 17 and a second nonionic polyoxyethylene ether with a HLB of about 10 to about 12. The composition further includes a glycol in a quantity sufficient to prevent formation of crystals of salicylic acid or another hydroxy acid.

In general, a composition according to this embodiment of the present invention in which the hydroxy acid is salicylic acid comprises:

(1) water;

(2) salicylic acid in an effective quantity;

(3) lanolin oil in an effective quantity;

(4) a complex comprising Vitamin A and Vitamin D₃ in an effective quantity;

(5) an emulsifier component in a quantity sufficient to effectively emulsify oil and water phases, the emulsifier component comprising:

(a) a first nonionic polyoxyethylene ether with a HLB of about to about 17; and

(b) a second nonionic polyoxyethylene ether with a HLB of about 10 to about 12;

(6) a glycol in a quantity sufficient to prevent formation of salicylic acid crystals; and

(7) optionally, allantoin in an effective quantity.

Preferably, the first nonionic polyoxyethylene ether is ceteareth-25 as described above.

Preferably, the second nonionic polyoxyethylene ether is ceteareth-6 with stearyl alcohol. A suitable formulation of ceteareth-6 with stearyl alcohol is marketed as “Cremophor A6.”

Typically, the glycol is selected from the group consisting of propylene glycol, ethoxydiglycol, and mixtures thereof In one preferred embodiment, the glycol is propylene glycol. In another preferred embodiment, the glycol is ethoxydiglycol. In yet another preferred embodiment, the glycol is a mixture of propylene glycol and ethoxydiglycol. Alternatively, other glycols can be used.

Optionally, the composition can further comprise additional ingredients.

The composition typically further comprises dimethicone.

Optionally, the composition may comprise at least one plant or herbal extract selected from the group consisting of arnica extract, St. John's wort extract, chamomile extract, and witch hazel extract.

Typically, the composition further comprises a long-chain fatty acid ester of glycerol selected from the group consisting of glyceryl stearate, glyceryl palmitate, and glyceryl arachidate. Preferably, the long-chain fatty acid ester of glycerol is glyceryl stearate.

Typically, the composition further comprises a lipid-soluble long-chain alcohol component. Preferably, the lipid-soluble long-chain alcohol component comprises at least one long-chain lipid-soluble alcohol selected from the group consisting of stearyl alcohol and cetyl alcohol. More preferably, the lipid-soluble long-chain alcohol comprises stearyl alcohol and cetyl alcohol.

Typically, the composition further comprises caprylic/capric triglycerides.

Typically, the composition further comprises a preservative component. Preferably, the preservative component comprises at least one preservative selected from the group consisting of propylparaben, methylparaben, and 2-methyl-4-isothiazolin-3-one. More preferably, the preservative component comprises propylparaben, methylparaben, and 2-methyl-4-isothiazolin-3-one. The 2-methyl-4-isothiazolin-3-one can also be replaced with a complex comprising methylchlorolisothiazolinone and 2-methyl-4-isothiazolin-3-one, marketed by Rohm & Haas as Kathon. Other preservatives known to cosmetic and drug formulators are also permissible.

Typically, the composition further comprises an antioxidant component. Preferably, the antioxidant component comprises at least one antioxidant selected from the group consisting of butylated hydroxytoluene, butylated hydroxyanisole, and a complex comprising propyl gallate, propylene glycol, and citric acid. Most preferably, the antioxidant component comprises butylated hydroxytoluene.

Typically, the composition further comprises a chelator component. A preferred chelator component is tetrasodium ethylenediaminetetraacetic acid (tetrasodium EDTA).

Typically, the composition further comprises fragrance. As indicated above, fragrance can be omitted.

The following gives percentages for the ingredients, including the optional ingredients.

Water can comprise from about 40.00% to about 70.00% of this embodiment of the composition. Preferably, water comprises about 50.06% of this embodiment of the composition.

Propylene glycol can comprise from about 15.0% to about 25.0% of this embodiment of the composition. Preferably, propylene glycol comprises about 20.00% of this embodiment of the composition.

Tetrasodium ethylenediaminetetraacetic acid can comprise from about 0.05% to about 0.20% of this embodiment of the composition. Preferably, tetrasodium ethylenediaminetetraacetic acid comprises about 0.15% of this embodiment of the composition.

Ceteareth-25 can comprise from about 1.50% to about 4.00% of this embodiment of the composition. Preferably, ceteareth-25 comprises about 3.20% of this embodiment of the composition.

Lanolin oil can comprise from about 3.00% to about 10.00% of this embodiment of the composition. Preferably, lanolin oil comprises about 7.00% of this embodiment of the composition.

Caprylic/capric triglycerides can comprise from about 1.00% to about 4.00% of this embodiment of the composition. Preferably, caprylic/capric triglycerides comprise about 2.00% of this embodiment of the composition.

Dimethicone can comprise from about 0.50% to about 3.00% of this embodiment of the composition. Preferably, dimethicone comprises about 1.40% of this embodiment of the composition.

Cetyl alcohol can comprise from about 3.00% to about 7.00% of this embodiment of the composition. Preferably, cetyl alcohol comprises about 4.10% of this embodiment of the composition.

Stearyl alcohol can comprise from about 2.00% to about 5.00% of this embodiment of the composition. Preferably, stearyl alcohol comprises about 2.90% of this embodiment of the composition.

Glyceryl stearate can comprise from about 1.00% to about 3.00% of this embodiment of the composition. Preferably, glyceryl stearate comprises about 2.20% of this embodiment of the composition.

Ceteareth-6 with stearyl alcohol can comprise from about 2.00% to about 4.00% of this embodiment of the composition. Preferably, ceteareth-6 with stearyl alcohol comprises about 3.00% of this embodiment of the composition.

Salicylic acid can comprise from about 1.80% to about 3.00% of this embodiment of the composition. Preferably, salicylic acid comprises about 2.00% of this embodiment of the composition.

Butylated hydroxytoluene can comprise from about 0.05% to about 2.00% of this embodiment of the composition. Preferably, butylated hydroxytoluene comprises about 0.10% of this embodiment of the composition.

The complex comprising Vitamin A and Vitamin D₃ can comprise from about 0.005% to about 0.50% of this embodiment of the composition. Preferably, the complex comprising Vitamin A and Vitamin D₃ comprises about 0.01% of this embodiment of the composition.

Methylparaben can comprise from about 0.10% to about 0.50% of this embodiment of the composition. Preferably, methylparaben comprises about 0.30% of this embodiment of the composition.

Propylparaben can comprise from about 0.10% to about 0.50% of this embodiment of the composition. Preferably, propylparaben comprises about 0.25% of this embodiment of the composition.

The preservative 2-methyl-4-isothiazolin-3-one can comprise from about 0.01% to about 0.08% of this embodiment of the composition. Preferably, 2-methyl-4-isothiazolin-3-one comprises about 0.03% of this embodiment of the composition.

At least one plant or herbal extracts, if present, can comprise from about 0.025% to about 0.80% of this embodiment of the composition.

Fragrance can comprise from about 0.10% to about 0.50% of this embodiment of the composition. Preferably, fragrance comprises about 0.30% of this embodiment of the composition.

Allantoin, if present, can comprise from about 0.01% to about 1.50% of this embodiment of the composition.

A preferred composition according to this embodiment of the invention comprises:

(1) water;

(2) salicylic acid in an effective quantity;

(3) lanolin oil in an effective quantity;

(4) an emulsifier component in a quantity sufficient to effectively emulsify oil and water phases, the emulsifier component comprising:

(a) ceteareth-25; and

(b) ceteareth-6 with stearyl alcohol;

(5) propylene glycol in a quantity sufficient to prevent formation of salicylic acid crystals;

(6) tetrasodium ethylenediaminetetraacetic acid;

(7) caprylic/capric triglycerides;

(8) dimethicone;

(9) cetyl alcohol;

(10) stearyl alcohol;

(11) glyceryl stearate;

(12) butylated hydroxytoluene;

(13) a complex comprising Vitamin A and Vitamin D₃;

(14) methylparaben;

(15) propylparaben;

(16) 2-methyl-4-isothiazolin-3-one;

(17) fragrance; and

(18) optionally, allantoin in an effective quantity.

A preferred composition according to this embodiment of the present invention comprises:

(1) from about 40.0% to about 70.0% of water;

(2) from about 15.0% to about 25.0% of propylene glycol;

(3) from about 0.05% to about 0.20% of tetrasodium ethylenediaminetetraacetic acid;

(4) from about 1.50% to about 4.00% of ceteareth-25;

(5) from about 3.00% to about 10.00% of lanolin oil;

(6) from about 1.00% to about 4.00% of caprylic/capric triglycerides;

(7) from about 0.50% to about 3.00% of dimethicone;

(8) from about 3.00% to about 7.00% of cetyl alcohol;

(9) from about 2.00% to about 5.00% of stearyl alcohol;

(10) from about 1.00% to about 3.00% of glyceryl stearate;

(11) from about 2.00% to about 4.00% of ceteareth-6 with stearyl alcohol;

(12) from about 1.80% to about 3.00% of salicylic acid;

(13) from about 0.05% to about 2.0% of butylated hydroxytoluene;

(14) from about 0.005% to about 0.50% of a complex comprising Vitamin A and Vitamin D₃;

(15) from about 0.10% to about 0.50% of methylparaben;

(16) from about 0.05% to about 0.30% of propylparaben;

(17) from about 0.01 % to about 0.06% of 2-methyl-4-isothiazolin-3-one; and

(18) from about 0.10% to about 0.50% of fragrance.

A preferred composition according to this embodiment of the invention comprises:

(1) about 50.06% of water;

(2) about 20.00% of propylene glycol;

(3) about 0.15% of tetrasodium ethylenediaminetetraacetic acid;

(4) about 3.20% of ceteareth-25;

(5) about 7.00% of lanolin oil;

(6) about 2.00% of caprylic/capric triglycerides;

(7) about 1.40% of dimethicone;

(8) about 4.10% of cetyl alcohol;

(9) about 2.90% of stearyl alcohol;

(10) about 2.20% of glyceryl stearate;

(11) about 3.00% of ceteareth-6 with stearyl alcohol;

(12) about 2.00% of salicylic acid;

(13) about 0.10% of butylated hydroxytoluene;

(14) about 0.01 % of a complex comprising Vitamin A and Vitamin D₃;

(15) about 0.30% of methylparaben;

(16) about 0.25% of propylparaben;

(17) about 0.03% of 2-methyl-4-isothiazolin-3-one; and

(18) about 0.30% of fragrance.

The salicylic acid in this embodiment can be replaced with another hydroxy acid as described above. Alternatively, the other hydroxy acid can be included along with salicylic acid. In the event that another hydroxy acid is used, the glycol is present in a quantity sufficient to prevent formation of crystals of the hydroxy acid or hydroxy acids.

C. Composition Using Two Emulsifiers. Glycol, and Hydrophilic Carbohydrate-Containing Polymer

Another embodiment of the present invention is a composition that uses two emulsifiers and a glycol as described above, but further comprises a hydrophilic carbohydrate-containing polymer.

In general, a composition according to this embodiment of the present invention comprises:

(1) water;

(2) salicylic acid in an effective quantity;

(3) lanolin oil in an effective quantity;

(4) a complex comprising Vitamin A and Vitamin D₃ in an effective quantity;

(5) an emulsifier component in a quantity sufficient to effectively emulsify oil and water phases, the emulsifier component comprising:

(a) a first nonionic polyoxyethylene ether with a HLB of about 15 to about 17 and;

(b) a second nonionic polyoxyethylene ether with a HLB of about 10 to about 12;

(6) at least one glycol in a quantity sufficient to prevent formation of salicylic acid crystals;

(7) a hydrophilic carbohydrate-containing polymer in an effective quantity; and

(8) optionally, allantoin in an effective quantity.

The glycol is as described above, and can be propylene glycol, ethoxydiglycol, or a mixture of propylene glycol and ethoxydiglycol.

Typically, the hydrophilic carbohydrate-containing polymer is selected from the group consisting of hydroxypropyl methylcellulose, hydroxyethyl ethylcellulose, methylcellulose, ethylcellulose, acacia gum, tragacanth gum, locust bean gum, guar gum, gum arabic, xanthan gum, and agarose. Preferably, the hydrophilic carbohydrate-containing polymer is hydroxypropyl methylcellulose.

The salicylic acid in this embodiment can be replaced with another hydroxy acid as described above. Alternatively, the other hydroxy acid can be included along with salicylic acid. In the event that another hydroxy acid is used, the glycol is present in a quantity sufficient to prevent formation of crystals of the hydroxy acid or hydroxy acids.

Optionally, the composition can further comprise additional ingredients.

The composition typically further comprises dimethicone.

Optionally, the composition may further comprise at least one plant or herbal extract, which may be selected from the group consisting of arnica extract, St. John's wort extract, chamomile extract, and witch hazel extract.

Typically, the composition further comprises a long-chain fatty acid ester of glycerol selected from the group consisting of glyceryl stearate, glyceryl palmitate, and glyceryl arachidate. Preferably, the long-chain fatty acid ester of glycerol is glyceryl stearate.

Typically, the composition further comprises a lipid-soluble long-chain alcohol component. Preferably, the lipid-soluble long-chain alcohol component comprises at least one long-chain lipid-soluble alcohol selected from the group consisting of stearyl alcohol and cetyl alcohol. More preferably, the lipid-soluble long-chain alcohol comprises stearyl alcohol and cetyl alcohol.

Typically, the composition further comprises caprylic/capric triglycerides.

Typically, the composition further comprises a preservative component. Preferably, the preservative component comprises at least one preservative selected from the group consisting of propylparaben, methylparaben, and 2-methyl-4-isothiazolin-3-one. More preferably, the preservative component comprises propylparaben, methylparaben, and 2-methyl-4-isothiazolin-3-one. The 2-methyl-4-isothiazolin-3-one can also be replaced with a complex comprising methylchlorolisothiazolinone and 2-methyl-4-isothiazolin-3-one.

Typically, the composition further comprises an antioxidant component. Preferably, the antioxidant component comprises at least one antioxidant selected from the group consisting of butylated hydroxytoluene, butylated hydroxyanisole, and a complex comprising propyl gallate, propylene glycol, and citric acid. Most preferably, the antioxidant component comprises butylated hydroxytoluene.

Typically, the composition further comprises a chelator component. A preferred chelator component is tetrasodium ethylenediaminetetraacetic acid (tetrasodium EDTA).

Typically, the composition further comprises fragrance. As indicated above, fragrance can be omitted.

In one alternative of this embodiment, the glycol is propylene glycol.

The following gives percentages for the ingredients, including the optional ingredients, for this alternative of the embodiment.

Water can comprise from about 40.0% to about 70.0% of this alternative of this embodiment of the composition. Preferably, water comprises about 53.89% of this alternative of this embodiment of the composition.

Propylene glycol can comprise from about 15.0% to about 30.0% of this alternative of this embodiment of the composition. Preferably, propylene glycol comprises about 18.0% of this alternative of this embodiment of the composition.

Tetrasodium ethylenediaminetetraacetic acid can comprise from about 0.005% to about 0.20% of this alternative of this embodiment of the composition. Preferably, tetrasodium ethylenediaminetetraacetic acid comprises about 0.15% of this alternative of this embodiment of the composition.

Ceteareth-25 can comprise from about 1.50% to about 4.00% of this alternative of this embodiment of the composition. Preferably, ceteareth-25 comprises about 3.20% of this alternative of this embodiment of the composition.

Hydroxypropyl methylcellulose can comprise from about 0.05% to about 0.30% of this alternative of this embodiment of the composition. Preferably, hydroxypropyl methylcellulose comprises about 0.16% of this alternative of this embodiment of the composition.

Lanolin oil can comprise from about 3.00% to about 10.00% of this alternative of this embodiment of the composition. Preferably, lanolin oil comprises about 7.00% of this alternative of this embodiment of the composition.

Caprylic/capric triglycerides can comprise from about 1.00% to about 4.00% of this alternative of this embodiment of the composition. Preferably, caprylic/capric triglycerides comprise about 2.00% of this alternative of this embodiment of the composition.

Dimethicone can comprise from about 0.50% to about 3.00% of this alternative of this embodiment of the composition. Preferably, dimethicone comprises about 1.40% of this alternative of this embodiment of the composition.

Cetyl alcohol can comprise from about 3.00% to about 7.00% of this alternative of this embodiment of the composition. Preferably, cetyl alcohol comprises about 3.60% of this alternative of this embodiment of the composition.

Stearyl alcohol can comprise from about 2.00% to about 5.00% of this alternative of this embodiment of the composition. Preferably, stearyl alcohol comprises about 2.60% of this alternative of this embodiment of the composition.

Ceteareth-6 with stearyl alcohol can comprise from about 2.00% to about 4.00% of this alternative of this embodiment of the composition. Preferably, ceteareth-6 with stearyl alcohol comprises about 3.00% of this alternative of this embodiment of the composition.

Glyceryl stearate can comprise from about 1.00% to about 4.00% of this alternative of this embodiment of the composition. Preferably, glyceryl stearate comprises about 2.20% of this alternative of this embodiment of the composition.

Salicylic acid can comprise from about 1.80% to about 3.00% of this alternative of this embodiment of the composition. Preferably, salicylic acid comprises about 2.00% of this alternative of this embodiment of the composition.

Butylated hydroxytoluene can comprise from about 0.05% to about 2.00% of this alternative of this embodiment of the composition. Preferably, butylated hydroxytoluene comprises about 0.10% of this alternative of this embodiment of the composition.

The complex comprising Vitamin A and Vitamin D₃ can comprise from about 0.005% to about 0.50% of this alternative of this embodiment of the composition. Preferably, the complex comprising Vitamin A and Vitamin D₃ comprises about 0.01 % of this alternative of this embodiment of the composition.

Methylparaben can comprise from about 0.10% to about 0.50% of this alternative of this embodiment of the composition. Preferably, methylparaben comprises about 0.25% of this alternative of this embodiment of the composition.

Propylparaben can comprise from about 0.05% to about 0.40% of this alternative of this embodiment of the composition. Preferably, propylparaben comprises about 0.20% of this alternative of this embodiment of the composition.

The preservative 2-methyl-4-isothiazolin-3-one can comprise from about 0.015 to about 0.06% of this alternative of this embodiment of the composition. Preferably, 2-methyl-4-isothiazolin-3-one comprises about 0.04% of this alternative of this embodiment of the composition.

At least one plant or herbal extract, if present, can comprise from about 0.025% to about 0.50% of this alternative of this embodiment of the composition.

Fragrance can comprise from about 0.20% to about 0.50% of this alternative of this embodiment of the composition. Preferably, fragrance comprises about 0.20% of this alternative of this embodiment of the composition.

Allantoin, if present, can comprise from about 0.01% to about 1.50% of this alternative of this embodiment of the composition.

A preferred composition according to this alternative of this embodiment of the invention comprises:

(1) water;

(2) salicylic acid in an effective quantity;

(3) lanolin oil in an effective quantity;

(4) an emulsifier component in a quantity sufficient to effectively emulsify oil and water phases, the emulsifier component comprising:

(a) ceteareth-25; and

(b) ceteareth-6 with stearyl alcohol;

(5) propylene glycol in a quantity sufficient to prevent formation of salicylic acid crystals;

(6) hydroxypropyl methylcellulose in an effective quantity;

(7) tetrasodium ethylenediaminetetraacetic acid;

(8) caprylic/capric triglycerides;

(9) dimethicone;

(10) cetyl alcohol;

(11) stearyl alcohol;

(12) glyceryl stearate;

(13) butylated hydroxytoluene;

(14) a complex comprising Vitamin A and Vitamin D₃;

(15) methylparaben;

(16) propylparaben;

(17) 2-methyl-4-isothiazolin-3-one;

(18) fragrance; and

(19) optionally, allantoin in an effective quantity.

A preferred composition according to this alternative of this embodiment of the invention comprises:

(1) from about 40.0% to about 70.0% of water;

(2) from about 15.0% to about 30.0% of propylene glycol;

(3) from about 0.005% to about 0.20% of tetrasodium ethylenediaminetetraacetic acid;

(4) from about 1.50% to about 4.00% of ceteareth-25;

(5) from about 0.05% to about 0.30% of hydroxypropyl methylcellulose;

(6) from about 3.00% to about 10.0% of lanolin oil;

(7) from about 1.00% to about 4.00% of caprylic/capric triglycerides;

(8) from about 0.50% to about 3.00% of dimethicone;

(9) from about 3.00% to about 7.00% of cetyl alcohol;

(10) from about 2.00% to about 5.00% of stearyl alcohol;

(11) from about 2.00% to about 4.00% of ceteareth-6 with stearyl alcohol;

(12) from about 1.00% to about 4.00% of glyceryl stearate;

(13) from about 1.80% to about 3.00% of salicylic acid;

(14) from about 0.05% to about 2.00% of butylated hydroxytoluene;

(15) from about 0.005% to about 0.50% of a complex comprising Vitamin A and Vitamin D3;

(16) from about 0.10% to about 0.50% of methylparaben;

(17) from about 0.05% to about 0.40% of propylparaben;

(18) from about 0.01 % to about 0.06% of 2-methyl-4-isothiazolin-3-one; and

(19) from about 0.10% to about 0.50% of fragrance.

A preferred composition according to this alternative of this embodiment of the invention comprises:

(1) about 53.89 % of water;

(2) about 18.00% of propylene glycol;

(3) about 0.15% of tetrasodium ethylenediaminetetraacetic acid;

(4) about 3.20% of ceteareth-25;

(5) about 0.16% of hydroxypropyl methylcellulose;

(6) about 7.00% of lanolin oil;

(7) about 2.00% of caprylic/capric triglycerides;

(8) about 1.40% of dimethicone;

(9) about 3.60% of cetyl alcohol;

(10) about 2.60% of stearyl alcohol;

(11) about 3.00% of ceteareth-6 with stearyl alcohol;

(12) about 2.20% of glyceryl stearate;

(13) about 2.00% of salicylic acid;

(14) about 0.10% of butylated hydroxytoluene;

(15) about 0.01 % of a complex comprising Vitamin A and Vitamin D3;

(16) about 0.25% of methylparaben;

(17) about 0.20% of propylparaben;

(18) about 0.04% of 2-methyl-4-isothiazolin-3-one; and

(19) about 0.20% of fragrance.

In one alternative of this embodiment, the glycol is a mixture of propylene glycol and ethoxydiglycol.

The following gives percentages for the ingredients, including the optional ingredients, for this alternative of the embodiment.

Water can comprise from about 40.0% to about 70.0% of this alternative of this embodiment of the composition. Preferably, water comprises about 52.94 % of this alternative of this embodiment of the composition.

Propylene glycol can comprise from about 5.00% to about 20.00% of this alternative of this embodiment of the composition. Preferably, propylene glycol comprises about 20.00% of this alternative of this embodiment of the composition.

Tetrasodium ethylenediaminetetraacetic acid can comprise from about 0.05% to about 2.00% of this alternative of this embodiment of the composition. Preferably, tetrasodium ethylenediaminetetraacetic acid comprises about 0.15% of this alternative of this embodiment of the composition.

Ceteareth-25 can comprise from about 1.50% to about 4.00% of this alternative of this embodiment of the composition. Preferably, ceteareth-25 comprises about 3.20% of this alternative of this embodiment of the composition.

Hydroxypropyl methylcellulose can comprise from about 0.05% to about 0.30% of this alternative of this embodiment of the composition. Preferably, hydroxypropyl methylcellulose comprises about 0.16% of this alternative of this embodiment of the composition.

Ethoxydiglycol, if present, can comprise from about 1.00% to about 6.00% of this alternative of this embodiment of the composition. Preferably, if the concentration of propylene glycol is at least 20%, ethoxydiglycol, if present, comprises no more than about 1.00% of this alternative of this embodiment of the composition.

Lanolin oil can comprise from about 3.00% to about 10.00% of this alternative of this embodiment of the composition. Preferably, lanolin oil comprises about 7.00% of this alternative of this embodiment of the composition.

Caprylic/capric triglycerides can comprise from about 1.00% to about 4.00% of this alternative of this embodiment of the composition. Preferably, caprylic/capric triglycerides comprise about 2.00% of this alternative of this embodiment of the composition.

Dimethicone can comprise from about 0.50% to about 3.00% of this alternative of this embodiment of the composition. Preferably, dimethicone comprises about 1.40% of this alternative of this embodiment of the composition.

Cetyl alcohol can comprise from about 3.00% to about 7.00% of this alternative of this embodiment of the composition. Preferably, cetyl alcohol comprises about 3.10% of this alternative of this embodiment of the composition.

Stearyl alcohol can comprise from about 2.00% to about 5.00% of this alternative of this embodiment of the composition. Preferably, stearyl alcohol comprises about 2.05% of this alternative of this embodiment of the composition.

Ceteareth-6 with stearyl alcohol can comprise from about 2.00% to about 5.00% of this alternative of this embodiment of the composition. Preferably, ceteareth-6 with stearyl alcohol comprises about 3.00% of this alternative of this embodiment of the composition.

Glyceryl stearate can comprise from about 1.00% to about 4.00% of this alternative of this embodiment of the composition. Preferably, glyceryl stearate comprises about 2.20% of this alternative of this embodiment of the composition.

Salicylic acid can comprise from about 1.80% to about 3.00% of this alternative of this embodiment of the composition. Preferably, salicylic acid comprises about 2.00% of this alternative of this embodiment of the composition.

Butylated hydroxytoluene can comprise from about 0.05% to about 0.90% of this alternative of this embodiment of the composition. Preferably, butylated hydroxytoluene comprises about 0.10% of this alternative of this embodiment of the composition.

The complex comprising Vitamin A and Vitamin D₃ can comprise from about 0.005% to about 0.50% of this alternative of this embodiment of the composition. Preferably, the complex comprising Vitamin A and Vitamin D₃ comprises about 0.01 % of this alternative of this embodiment of the composition.

Methylparaben can comprise from about 0.10% to about 0.50% of this alternative of this embodiment of the composition. Preferably, methylparaben comprises about 0.25% of this alternative of this embodiment of the composition.

Propylparaben can comprise from about 0.10% to about 0.50% of this alternative of this embodiment of the composition. Preferably, propylparaben comprises about 0.20% of this alternative of this embodiment of the composition.

The preservative 2-methyl-4-isothiazolin-3-one can comprise from about 0.01% to about 0.06% of this alternative of this embodiment of the composition. Preferably, 2-methyl-4-isothiazolin-3-one comprises about 0.04% of this alternative of this embodiment of the composition.

Fragrance can comprise from about 0.10% to about 0.50% of this alternative of this embodiment of the composition. Preferably, fragrance comprises about 0.20% of this alternative of this embodiment of the composition.

At least one plant or herbal extract, if present, can comprise from about 0.025% to about 0.50% of this alternative of this embodiment of the composition.

Allantoin, if present, can comprise from about 0.01% to about 1.50% of this alternative of this embodiment of the composition.

A preferred composition according to this alternative of this embodiment of the invention comprises:

(1) water;

(2) salicylic acid in an effective quantity;

(3) lanolin oil in an effective quantity;

(4) an emulsifier component in a quantity sufficient to effectively emulsify oil and water phases, the emulsifier component comprising:

(a) ceteareth-25; and

(b) ceteareth-6 with stearyl alcohol;

(5) propylene glycol;

(6) optionally, ethoxydiglycol, the propylene glycol and ethoxydiglycol being present in a quantity sufficient to prevent formation of salicylic acid crystals;

(7) hydroxypropyl methylcellulose in an effective quantity;

(8) tetrasodium ethylenediaminetetraacetic acid;

(9) caprylic/capric triglycerides;

(10) dimethicone;

(11) cetyl alcohol;

(12) stearyl alcohol;

(13) glyceryl stearate;

(14) butylated hydroxytoluene;

(15) a complex comprising Vitamin A and Vitamin D₃;

(16) methylparaben;

(17) propylparaben;

(18) 2-methyl-4-isothiazolin-3-one;

(19) fragrance; and

(20) optionally, allantoin in an effective quantity

A preferred composition according to this alternative of this embodiment of the invention comprises:

(1) from about 40.0% to 70.0% of water;

(2) from about 5.00% to 20.0% of propylene glycol;

(3) from about 0.05% to about 2.00% of tetrasodium ethylenediaminetetraacetic acid;

(4) from about 1.50% to about 4.00% of ceteareth-25;

(5) from about 0.05% to about 0.30% of hydroxypropyl methylcellulose;

(6) from about 3.00% to about 10.00% of lanolin oil;

(7) optionally, from about 1.00% to about 6.00% of ethoxydiglycol;

(8) from about 1.00% to about 4.00% of caprylic/capric triglycerides;

(9) from about 0.50% to abut 3.00% of dimethicone;

(10) from about 3.00% to about 7.00% of cetyl alcohol;

(11) from about 2.00% to about 5.00% of stearyl alcohol;

(12) from about 2.00% to about 5.00% of ceteareth-6 with stearyl alcohol;

(13) from about 1.00% to about 4.00% of glyceryl stearate;

(14) from about 1.80% to about 3.00% of salicylic acid;

(15) from about 0.05% to about 0.90% of butylated hydroxytoluene;

(16) from about 0.005% to about 0.50% of a complex comprising Vitamin A and Vitamin D₃;

(17) from about 0.10% to about 0.50% of methylparaben;

(18) from about 0.10% to about 0.50% of propylparaben;

(19) from about 0.01% to about 0.06% of 2-methyl-4-isothiazolin-3-one; and

(20) from about 0.10% to about 0.50% fragrance.

A preferred composition according to this alternative of this embodiment of the invention comprises:

(1) about 52.94% of water;

(2) about 20.00% of propylene glycol;

(3) about 0.15% of tetrasodium ethylenediaminetetraacetic acid;

(4) about 3.20% of ceteareth-25;

(5) about 0.16% of hydroxypropyl methylcellulose;

(6) about 7.00% of lanolin oil;

(7) optionally, about 1.00% of ethoxydiglycol;

(8) about 2.00% of caprylic/capric triglycerides;

(9) about 1.40% of dimethicone;

(10) about 3.10% of cetyl alcohol;

(11) about 2.05% of stearyl alcohol;

(12) about 3.00% of ceteareth-6 with stearyl alcohol;

(13) about 2.20% of glyceryl stearate;

(14) about 2.00% of salicylic acid;

(15) about 0.10% of butylated hydroxytoluene;

(16) about 0.01 % of a complex comprising Vitamin A and Vitamin D₃;

(17) about 0.25% of methylparaben;

(18) about 0.20% of propylparaben;

(19) about 0.04% of 2-methyl-4-isothiazolin-3-one; and

(20) about 0.20% of fragrance.

D. Composition Using Two Emulsifiers, Glycol, and Quaternary Ammonium-Containing Polymer

Another embodiment of the present invention is a composition that uses two emulsifiers and a glycol as described above, but further comprises a quaternary ammonium-containing polymer.

In general, a composition according to this embodiment of the present invention comprises:

(1) water;

(2) salicylic acid in an effective quantity;

(3) lanolin oil in an effective quantity;

(4) a complex comprising Vitamin A and Vitamin D₃ in an effective quantity;

(5) an emulsifier component in a quantity sufficient to effectively emulsify oil and water phases, the emulsifier component comprising:

(a) a first nonionic polyoxyethylene ether with a HLB of about 15 to about 17; and

(b) a second nonionic polyoxyethylene ether with a HLB of about 10 to about 12;

(6) at least one glycol in a quantity sufficient to prevent formation of salicylic acid crystals;

(7) a quaternary ammonium-containing polymer in an effective quantity; and

(8) optionally, allantoin in an effective quantity.

The glycol is as described above, and can be propylene glycol, ethoxydiglycol, or a mixture of propylene glycol and ethoxydiglycol. In one preferred alternative, the glycol is propylene glycol.

The salicylic acid in this embodiment can be replaced with another hydroxy acid as described above. Alternatively, the other hydroxy acid can be included along with salicylic acid. In the event that another hydroxy acid is used, the glycol is present in a quantity sufficient to prevent formation of crystals of the hydroxy acid or hydroxy acids.

Typically, the quaternary ammonium-containing polymer is polyquaternium-10.

Optionally, the composition can further comprise additional ingredients.

The composition typically further comprises dimethicone.

Optionally, the composition may further comprise at least one plant or herbal extract, which may be selected from the group consisting of arnica extract, St. John's wort extract, chamomile extract, and witch hazel extract.

Typically, the composition further comprises a long-chain fatty acid ester of glycerol selected from the group consisting of glyceryl stearate, glyceryl palmitate, and glyceryl arachidate. Preferably, the long-chain fatty acid ester of glycerol is glyceryl stearate.

Typically, the composition further comprises a lipid-soluble long-chain alcohol component. Preferably, the lipid-soluble long-chain alcohol component comprises at least one long-chain lipid-soluble alcohol selected from the group consisting of stearyl alcohol and cetyl alcohol. More preferably, the lipid-soluble long-chain alcohol comprises stearyl alcohol and cetyl alcohol.

Typically, the composition further comprises caprylic/capric triglycerides.

Typically, the composition further comprises a preservative component. Preferably, the preservative component comprises at least one preservative selected from the group consisting of propylparaben, methylparaben, and 2-methyl-4-isothiazolin-3-one. More preferably, the preservative component comprises propylparaben, methylparaben, and 2-methyl-4-isothiazolin-3-one. The 2-methyl-4-isothiazolin-3-one can also be replaced with a complex comprising methylchlorolisothiazolinone and 2-methyl-4-isothiazolin-3-one.

Typically, the composition further comprises an antioxidant component. Preferably, the antioxidant component comprises at least one antioxidant selected from the group consisting of butylated hydroxytoluene, butylated hydroxyanisole, and a complex comprising propyl gallate, propylene glycol, and citric acid. Most preferably, the antioxidant component comprises butylated hydroxytoluene.

Typically, the composition further comprises a chelator component. A preferred chelator component is tetrasodium ethylenediaminetetraacetic acid (tetrasodium EDTA).

Typically, the composition further comprises fragrance. As indicated above, fragrance can be omitted.

The following gives percentages for the ingredients, including the optional ingredients.

Water can comprise from about 40.00% to about 70.00% of this embodiment of the composition. Preferably, water comprises about 52.49% of this embodiment of the composition.

Propylene glycol can comprise from about 15.0% to about 30.0% of this embodiment of the composition. Preferably, propylene glycol comprises about 20.00% of this embodiment of the composition.

Tetrasodium ethylenediaminetetraacetic acid can comprise from about 0.05% to about 0.20% of this embodiment of the composition. Preferably, tetrasodium ethylenediaminetetraacetic acid comprises about 0.15% of this embodiment of the composition.

Ceteareth-25 can comprise from about 1.50% to about 3.50% of this embodiment of the composition. Preferably, ceteareth-25 comprises about 3.20% of this embodiment of the composition.

Polyquaternium-10 can comprise from about 0.05% to about 0.30% of this embodiment of the composition. Preferably, polyquaternium-10 comprises about 0.15% of this embodiment of the composition.

Lanolin oil can comprise from about 3.00% to about 10.00% of this embodiment of the composition. Preferably, lanolin oil comprises about 7.00% of this embodiment of the composition.

Caprylic/capric triglycerides can comprise from about 1.00% to about 4.00% of this embodiment of the composition. Preferably, caprylic/capric triglycerides comprise about 2.00% of this embodiment of the composition.

Dimethicone can comprise from about 0.50% to about 3.00% of this embodiment of the composition. Preferably, dimethicone comprises about 1.40% of this embodiment of the composition.

Cetyl alcohol can comprise from about 3.00% to about 7.00% of this embodiment of the composition. Preferably, cetyl alcohol comprises about 3.30% of this embodiment of the composition.

Stearyl alcohol can comprise from about 2.00% to about 5.00% of this embodiment of the composition. Preferably, stearyl alcohol comprises about 2.30% of this embodiment of the composition.

Ceteareth-6 with stearyl alcohol can comprise from about 2.00% to about 5.00% of this embodiment of the composition. Preferably, ceteareth-6 with stearyl alcohol comprises about 3.00% of this embodiment of the composition.

Glyceryl stearate can comprise from about 1.00% to about 3.00% of this embodiment of the composition. Preferably, glyceryl stearate comprises about 2.20% of this embodiment of the composition.

Salicylic acid can comprise from about 1.80% to about 3.00% of this embodiment of the composition. Preferably, salicylic acid comprises about 2.00% of this embodiment of the composition.

Butylated hydroxytoluene can comprise from about 0.05% to about 0.90% of this embodiment of the composition. Preferably, butylated hydroxytoluene comprises about 0.10% of this embodiment of the composition.

The complex comprising Vitamin A and Vitamin D₃ can comprise from about 0.005% to about 0.50% of this embodiment of the composition. Preferably, the complex comprising Vitamin A and Vitamin D₃ comprises about 0.01 % of this embodiment of the composition.

Methylparaben can comprise from about 0.10% to about 0.50% of this embodiment of the composition. Preferably, methylparaben comprises about 0.25% of this embodiment of the composition.

Propylparaben can comprise from about 0.10% to about 0.50% of this embodiment of the composition. Preferably, propylparaben comprises about 0.20% of this embodiment of the composition.

The preservative 2-methyl-4-isothiazolin-3-one can comprise from about 0.01% to about 0.06% of this embodiment of the composition. Preferably, 2-methyl-4-isothiazolin-3-one comprises about 0.04% of this embodiment of the composition.

Fragrance can comprise from about 0.10% to about 0.50% of this embodiment of the composition. Preferably, fragrance comprises about 0.20% of this embodiment of the composition.

At least one plant or herbal extract, if present, can comprise from about 0.025% to about 0.50% of this alternative of this embodiment of the composition.

Allantoin, if present, can comprise from about 0.01% to about 1.50% of this embodiment of the composition.

A preferred composition according to this embodiment of the invention comprises:

(1) water;

(2) salicylic acid in an effective quantity;

(3) lanolin oil in an effective quantity;

(4) an emulsifier component in a quantity sufficient to effectively emulsify oil and water phases, the emulsifier component comprising:

(a) ceteareth-25; and

(b) ceteareth-6 with stearyl alcohol;

(5) propylene glycol in a quantity sufficient to prevent formation of salicylic acid crystals;

(6) polyquaternium-10 in an effective quantity;

(7) tetrasodium ethylenediaminetetraacetic acid;

(8) caprylic/capric triglycerides;

(9) dimethicone;

(10) cetyl alcohol;

(11) stearyl alcohol;

(12) glyceryl stearate;

(13) butylated hydroxytoluene;

(14) a complex comprising Vitamin A and Vitamin D₃;

(15) methylparaben;

(16) propylparaben;

(17) 2-methyl-4-isothiazolin-3-one;

(18) fragrance; and

(19) optionally, allantoin in an effective quantity.

A more preferred composition according to this embodiment of the present invention comprises:

(1) from about 40.0% to about 70.0% of water;

(2) from about 15.0% to about 30.0% of propylene glycol;

(3) from about 0.05% to about 0.20% of tetrasodium ethylenediaminetetraacetic acid;

(4) from about 1.50% to about 3.50% of ceteareth-25;

(5) from about 0.05% to about 0.30% of polyquaternium-10;

(6) from about 3.00% to about 10.00% of lanolin oil;

(7) from about 1.00% to about 4.00% of caprylic/capric triglycerides;

(8) from about 0.50% to about 3.00% of dimethicone;

(9) from about 3.00% to about 7.00% of cetyl alcohol;

(10) from about 2.00% to about 5.00% of stearyl alcohol;

(11) from about 2.00% to about 5.00% of ceteareth-6 with stearyl alcohol;

(12) from about 1.00% to about 3.00% of glyceryl stearate;

(13) from about 1.80% to about 3.00% of salicylic acid;

(14) from about 0.05% to about 0.90% of butylated hydroxytoluene;

(15) from about 0.005% to about 0.50% of a complex comprising Vitamin A and Vitamin D3;

(16) from about 0.10% to about 0.50% of methylparaben;

(17) from about 0.10% to about 0.50% of propylparaben;

(18) from about 0.01 % to about 0.06% of 2-methyl-4-isothiazolin-3-one; and

(19) from about 0.10% to about 0.50% of fragrance.

A preferred composition according to this embodiment of the invention comprises:

(1) about 50.01 % of water;

(2) about 20.0% of propylene glycol;

(3) about 0.15% of tetrasodium ethylenediaminetetraacetic acid;

(4) about 3.20% of ceteareth-25;

(5) about 0.16% of polyquaternium-10;

(6) about 7.00% of lanolin oil;

(7) about 2.00% of caprylic/capric triglycerides;

(8) about 1.40% of dimethicone;

(9) about 4.10% of cetyl alcohol;

(10) about 2.90% of stearyl alcohol;

(11) about 3.00% of ceteareth-6 with stearyl alcohol;

(12) about 2.20% of glyceryl stearate;

(13) about 2.00% of salicylic acid;

(14) about 0.10% of butylated hydroxytoluene;

(15) about 0.01% of a complex comprising Vitamin A and Vitamin D₃;

(16) about 0.25% of methylparaben;

(17) about 0.20% of propylparaben;

(18) about 0.03% of 2-methyl-4-isothiazolin-3-one; and

(19) about 0.30% of fragrance.

In all of the embodiments of a composition according to the present invention described above, other ingredients can be used in addition to or in place of the ingredients recited above.

For example, other lipid-soluble components can be used in addition to or in place of the caprylic/capric triglycerides. Such components can include but are not limited to: steareth-2; steareth-21; polyglyceryl-3 beeswax; a branched-chain carboxylic acid ester of a branched-chain alcohol selected from the group consisting of isononyl isononanoate, isodecyl isononanoate, isooctyl isononanoate, isononyl isooctanoate, isodecyl isooctanoate, isooctyl isooctanoate, isononyl isodecanoate, isooctyl isodecanoate, and isodecyl isodecanoate; acrylates/C₁₀-C₃₀ alkyl acrylates cross-polymers; methylgluceth-20; a glyceryl ester of a long-chain fatty acid selected from the group consisting of glyceryl monostearate, glyceryl monopalmitate, and glyceryl monoarachidate; hydrogenated vegetable oil; squalane; C₁₂-C₁₅ alkylbenzoates; di-C₁₂-C₁₅ alkylfumarate; cholesterol; lanolin alcohol; octyldodecanol; isostearic acid; a branched-chain neopentanoate selected from the group consisting of octyldodecyl neopentanoate, heptyldodecyl neopentanoate, nonyldodecyl neopentanoate, octylundecyl neopentanoate, heptylundecyl neopentanoate, nonylundecyl neopentanoate, octyltridecyl neopentanoate, heptyltridecyl neopentanoate, and nonyltridecyl neopentanoate; an arachidyl ester of a short-chain carboxylic acid selected from the group consisting of arachidyl propionate, arachidyl acetate, arachidyl butyrate, and arachidyl isobutyrate; a long-chain fatty acid ester of a medium-chain alcohol selected from the group consisting of octyl palmitate, octyl myristate, octyl stearate, heptyl palmitate, heptyl myristate, heptyl stearate, nonyl palmitate, nonyl myristate, and nonyl stearate; jojoba oil; a myristyl ester of a long-chain fatty acid selected from the group consisting of myristyl myristate, myristyl laurate, and myristyl palmitate; bisabolol; hydrogenated jojoba oil; jojoba esters; methylgluceth-20 sesquistearate; PPG-14 butyl ether; PPG-15 stearyl ether; PPG-1-isoceteth-3-acetate; laureth-2-benzoate; diisostearyl dimer dilinoleate; a long-chain cis-monounsaturated fatty acid ester of a medium-chain alcohol; a medium-chain saturated carboxylic acid ester of a long-chain alcohol; hydrogenated soy glycerides; a long-chain fatty acid ester of cetyl alcohol selected from the group consisting of cetyl palmitate, cetyl stearate, and cetyl myristate; palm kernel oil; and palm oil.

Still other lipid-soluble compounds are known in the cosmetic art and can be used in addition to or in place of these compounds, including derivatives and analogues of these compounds.

In addition, the composition can further comprise other ingredients that are generally used in the cosmetic art and in the art of over-the-counter skin preparations. These ingredients include, but are not limited to:

(1) plant extracts, such as horsetail extract, horse chestnut extract, rose extract, or lavender extract;

(2) a short-chain carboxylic acid ester of tocopherol selected from the group consisting of tocopheryl acetate, tocopheryl propionate, tocopheryl butyrate, and tocopheryl isobutyrate;

(3) a long-chain fatty acid ester of ascorbic acid selected from the group consisting of ascorbyl myristate, ascorbyl palmitate, and ascorbyl stearate;

(4) a long-chain fatty acid ester of retinol or a retinol derivative or analogue wherein the acyl moiety of the ester is selected from the group consisting of myristic acid, palmitic acid, and stearic acid; and

(5) a sunscreen, which can be at least one compound selected from the group consisting of octyl methoxycinnamate, p-aminobenzoic acid, ethyl p-aminobenzoate, isobutyl p-aminobenzoate, glyceryl p-aminobenzoate, p-dimethylaminobenzoic acid, methyl anthranilate, menthyl anthranilate, phenyl anthranilate, benzyl anthranilate, phenylethyl anthranilate, linalyl anthranilate, terpinyl anthranilate, cyclohexenyl anthranilate, amyl salicylate, phenyl salicylate, benzyl salicylate, menthyl salicylate, glyceryl salicylate, dipropyleneglycol salicylate, methyl cinnamate, benzyl cinnamate, α-phenyl cinnamonitrile, butyl cinnamoylpyruvate, umbelliferone, methylacetoumbelliferone, esculetin, methylesculetin, daphnetin, esculin, daphnin, diphenylbutadiene, stilbene, dibenzalacetone, benzalacetophenone, sodium 2-naphthol-3,6-disulfonate, sodium 2-naphthol-6,8-disulfonate, dihydroxynaphthoic acid, salts of dihydroxynaphthoic acid, o-hydroxybiphenyldisulfonates, p-hydroxybiphenyldisulfonates, 7-hydroxycoumarin, 7-methylcoumarin, 3-phenylcoumarin, 2-acetyl-3-bromoindazole, phenylbenzoxazole, methylnaphthoxazole, arylbenzothiazoles, quinine bisulfate, quinine sulfate, quinine chloride, quinine oleate, quinine tannate, 8-hydroxyquinoline salts, 2-phenylquinoline, hydroxy-substituted benzophenones, methoxy-substituted benzophenones, uric acid, vilouric acid, tannic acid, tannic acid hexaethylether, hydroquinone, oxybenzone, sulisobenzone, dioxybenzone, benzoresorcinol, 2,2′,4,4′-tetrahydroxybenzophenone, 2,2′dihydroxy-4,4′-dimethoxybenzophenone, octabenzone, 4-isopropyldibenzoylmethane, butylmethoxydibenzoylmethane, etocrylene, and 4-isopropyldibenzoylmethane.

Other ingredients can also optionally be included, such as colorants, pigments, opacifiers, and the like, such as are conventionally used in cosmetics and over-the-counter skin preparations.

The composition can further include other ingredients that are conventional in the cosmetic art and the art of over-the-counter skin preparations.

Compositions according to the present invention can be prepared by standard mixing techniques, such as are conventional in the cosmetic art and in the art of over-the-counter drug formulation for blending lipid-soluble components and water-soluble components. These mixing techniques include both manual and mechanical mixing, and include homogenization mixing and propeller and sweep mixing. The mixing techniques to be used can be chosen by one of ordinary skill in the art based on variables such as the viscosity of the components to be mixed and the volumes of those components, as well as the relative proportions of lipid-soluble and water-soluble ingredients. The composition can be mixed in two or more batches, such as one batch containing lipid-soluble ingredients and another batch containing water-soluble ingredients, and the batches can then be mixed at the final stage of preparation.

In general, a preferred method for preparing compositions according to the present invention, the oil-in-water emulsion comprising a first nonionic polyoxyethylene ether with a HLB of about 15 to about 17 and a second nonionic polyoxyethylene ether with a HLB of about 10 to about 12, if present, is:

(1) preparing a first mixture comprising an aqueous phase including the first nonionic polyoxyethylene ether;

(2) preparing a second mixture comprising a lipid phase including the second nonionic polyoxyethylene ether, if present;

(3) combining the first and second mixtures at a temperature of about 160° F. to about 180° F. to form an emulsion;

(4) mixing the emulsion at a sufficiently high shear rate while cooling the emulsion to about 130° F. to about 160° F. so that the emulsion is stable during a transition from a water-in-oil emulsion to an oil-in-water emulsion upon cooling; and

(5) cooling the emulsion for storage.

Typically, the temperature at which step (3) occurs is from about 165° F. to about 175° F. Typically, the temperature at which step (4) occurs is about 155° F. Typically, the temperature at which step (5) occurs is from about 100° F. to about 115° F.

Other sequences can be used for mixing, depending on the number, quantity, and identity of the ingredients used.

### II. ENCAPSULATED COMPOSITIONS

Another aspect of the present invention is an encapsulated composition. In general, an encapsulated composition according to the present invention comprises:

(1) a shell comprising agar or gelatin;

(2) a solution of salicylic acid or another hydroxy acid as described above dissolved in at least one glycol, the solution being encapsulated in the shell; and

(3) a carrier for the shell, the carrier being in the form of a cream, gel, or ointment, the shell being broken to release the solution of salicylic acid when the composition is applied to the skin of a user.

The hydroxy acid can be salicylic acid or another α-hydroxy acid, β-hydroxy acid, or multi-hydroxy acid as described above.

The glycol can be propylene glycol, ethoxydiglycol, or a mixture of propylene glycol and ethoxydiglycol, as described above.

Typically, the carrier is in the form of a cream. Typically, the cream is an oil-in-water emulsion that contains other ingredients, such as a complex comprising Vitamin A and Vitamin B₃ and lanolin oil, as well as the other ingredients used in the compositions described above in Section (11), such as emulsifiers such as ceteareth-25 and ceteareth-6 with stearyl alcohol, glyceryl stearate, dimethicone, caprylic/capric triglycerides, cetyl alcohol, stearyl alcohol, preservatives such as methylparaben, propylparaben, and 2-methyl-4-isothiazolin-3-one, antioxidants such as butylated hydroxytoluene, chelators such as tetrasodium ethylenediaminetetraacetic acid, , fragrance, quaternary ammonium-containing polymers such as polyquaternium-10, and hydrophilic carbohydrate-containing copolymers such as hydroxypropyl methylcellulose. Other ingredients can be used, such as other emulsifiers.

Alternatively, the carrier can be in the form of an ointment or a gel. If the carrier is in the form of a gel, typically it includes one or more gel-forming ingredients, such as a complex comprising mineral oil and at least one hydrogenated alkylene copolymer; a polymer selected from the group consisting of polyglyceryl methacrylate, polyglyceryl acrylate, and polyglyceryl ethacrylate; or a carboxypolymethylene polymer. Other gel-forming ingredients can be used.

Typically, the carrier comprises about 70% by weight of the composition and the shell and encapsulated solution of salicylic acid comprise about 30% by weight of the composition.

Because the salicylic acid or other hydroxy acid is held within the capsule, it does not effect emulsion or fragrance stability. Delivery of the salicylic acid or other hydroxy acid is accomplished as the capsules are broken as the composition is rubbed into the skin.

### III. METHODS FOR TREATMENT OF DERMATOLOGICAL OR SYSTEMIC INFLAMMATORY CONDITIONS

Another aspect of the present invention is methods for the treatment of dermatological or systemic inflammatory conditions such as psoriasis, seborrheic dermatitis and dandruff. In general, these methods comprise applying a composition according to the present invention to the skin of a patient in a therapeutically effective quantity.

The dosages to be administered can be determined by one of ordinary skill in the art, depending on the clinical severity of the disease, the age and weight of the patient, the exposure of the patient to conditions that may precipitate outbreaks of psoriasis or other dermatological or systemic inflammatory conditions, the degree of exposure to such conditions as sunlight or tobacco smoke, and other pharmacokinetic factors generally understood in the art, such as liver and kidney metabolism. The interrelationship of dosages for animals of various sizes and species and humans based on mg/m3 of surface area is described by E. J. Freireich et al., “Quantitative Comparison of Toxicity of Anticancer Agents in Mouse, Rat, Hamster, Dog, Monkey, and Man,” Cancer Chemother. Rep. 50: 219-244 (1966). Adjustments in the dosage regimen can be made to optimize the therapeutic response. Doses can be divided and administered on a daily basis or the dose can be reduced proportionally depending upon the therapeutic situation.

Compositions according to the present invention can be administered alone or together with other topically or systemically administered agents for the treatment of psoriasis. These agents can include, but are not limited to, calcipotriene, anthralin, psoralen, 8-methoxypsoralen, 5-methoxypsoralen, 4,5,8-trimethylpsoralen, coal tar, antihistamines, aminopterin, methotrexate, azathioprine, hydroxyurea, 6-thioguanine, cyclosporine, sulfasalazine, etretinate, isotretinoin, and colchicine. Other agents can be used. These agents are administered by methods that are well known in the art and need not be detailed further here.

This invention is described by the following Examples. These Examples are provided for illustrative purposes only and are not intended to limit the invention.

### EXAMPLES

### Example 1

### Skin Care Composition Containing Salicylic Acid and Ceteareth-25 as Emulsifier

A skin care composition according to the present invention containing salicylic acid and ceteareth-25 as emulsifier is prepared according to Table 1.

TABLE 1
SKIN CARE COMPOSITION CONTAINING SALICYLIC ACID
AND CETEARETH-25 AS EMULSIFIER

| | CONCENTRATION | PREFERRED |
|---|---|---|
| INGREDIENT | RANGE, % | CONCENTRATION % |
| Water Phase | | |
| Water | 40.0-80.0 | 45.0-60.0 |
| Tetrasodium EDTA | 0.05-0.50 | 0.10-0.30 |
| Propylene Glycol | 2.0-10.0 | 3.0-6.0 |
| Ceteareth-25 | 1.0-6.0 | 1.5-3.5 |
| Hydroxypropyl | 0.05-0.50 | 0.15-0.30 |
| methylcellulose | | |
| Oil Phase | | |
| Lanolin Oil | 2.0-20.0 | 5.0-12.0 |
| Caprylic/Capric | 0.5-5.0 | 1.0-3.0 |
| Triglycerides | | |
| Dimethicone | 0.5-5.0 | 1.0-2.0 |
| Cetyl Alcohol | 2.0-12.0 | 3.0-6.0 |
| Stearyl Alcohol | 1.0-10.0 | 2.0-4.0 |
| Salicylic Acid | 1.8-3.0 | 2.0-2.5 |
| Butylated | 0.01-1.0 | 0.08-0.50 |
| Hydroxytoluene | | |
| Glyceryl Stearate | 1.0-5.0 | 1.5-3.5 |
| Other Ingredients | | |
| Methylparaben | 0.10-0.50 | 0.20-0.40 |
| Propylparaben | 0.10-0.50 | 0.20-0.40 |
| 2-Methyl-4- | 0.01-0.05 | 0.02-0.04 |
| isothiazolin-3-one | | |
| Arnica Extract | 0.025-0.50 | 0.025-0.25 |
| St. John's Wort | 0.025-0.50 | 0.025-0.25 |
| Extract | | |
| Chamomile Extract | 0.025-0.50 | 0.025-0.25 |
| Witch Hazel Extract | 0.025-0.50 | 0.025-0.25 |
| Fragrance | 0.10-0.50 | 0.20-0.40 |

The water phase and oil phase are heated separately to about 170-180° F. while each is being adequately stirred. The melted uniform oil phase is then added to the heated uniform water phase. This is allowed to be adequately mixed and homogenized prior to cooling the emulsion to 120-125° F. At this point, the other ingredients are added, mixed thoroughly, and then cooled to 105-110° F. prior to filling and packaging. The oil-in-water cream that is produced is stable but with time salicylic acid crystals are formed as they precipitate out of the emulsion. These crystals remain uniformly dispersed while in suspension and may damage weak skin when the cream is rubbed in.

### Example 2

### Skin Care Composition Containing Salicylic Acid Two Emulsifiers and High Proportion of Propylene Glycol

A skin care composition according to the present invention containing salicylic acid, two emulsifiers, and a high proportion of propylene glycol is prepared according to Table 2. It was found that salicylic acid shard formation could be prevented by raising the propylene glycol concentration to about 20%.

The water phase and oil phase are heated separately to 170-180° F. while being stirred and them combined and mixed, after which the emulsion is cooled to about 125° F. and the other ingredients are added and mixed. Although no shards of salicylic acid formed with time, the emulsion stability, when heated to 40° C. under accelerated aging, was found to be lacking. Separation of the emulsion occurred after about five weeks at 40° C.

TABLE 2
SKIN CARE COMPOSITION CONTAINING SALICYLIC ACID,
TWO EMULSIFIERS AND HIGH PROPORTION OF PROPYLENE
GLYCOL

| | CONCENTRATION | PREFERRED |
|---|---|---|
| INGREDIENT | RANGE. % | CONCENTRATION % |
| Water Phase | | |
| Water | 40.0-70.0 | 50.06 |
| Propylene Glycol | 15.0-25.0 | 20.00 |
| Tetrasodium EDTA | 0.05-0.20 | 0.15 |
| Ceteareth-25 | 1.50-4.00 | 3.20 |
| Oil Phase | | |
| Lanolin Oil | 3.00-10.00 | 7.00 |
| Caprylic/Capric | 1.00-4.00 | 2.00 |
| Triglycerides | | |
| Dimethicone | 0.50-3.00 | 1.40 |
| Cetyl Alcohol | 3.00-7.00 | 4.10 |
| Stearyl Alcohol | 2.00-5.00 | 2.90 |
| Glyceryl Stearate | 1.00-3.00 | 2.20 |
| Ceteareth-6 with | 2.00-4.00 | 3.00 |
| Stearyl Alcohol | | |
| Salicylic Acid | 1.80-3.00 | 2.00 |
| Butylated | 0.05-2.00 | 0.10 |
| Hydroxytoluene | | |
| Cod Liver Oil | 0.05-2.00 | 0.10 |
| Vitamin A and | 0.005-0.50 | 0.01 |
| Vitamin D₃ | | |
| Other Ingredients | | |
| Methylparaben | 0.10-0.50 | 0.30 |
| Propylparaben | 0.05-0.30 | 0.25 |
| 2-Methyl-4- | 0.01-0.06 | 0.03 |
| isothiazolin-3-one | | |
| Arnica Extract | 0.025-0.25 | 0.10 |
| St. John's Wort | 0.025-0.25 | 0.10 |
| Extract | | |
| Chamomile Extract | 0.025-0.25 | 0.10 |
| Witch Hazel Extract | 0.025-0.25 | 0.10 |
| Fragrance | 0.10-0.50 | 0.30 |

### Example 3

### Skin Care Composition Containing Salicylic Acid, Two Emulsifiers

### High Proportion of Propylene Glycol, and Hydroxypropyl Methylcellulose

A skin care composition according to the present invention containing salicylic acid, two emulsifiers, a high proportion of propylene glycol, and the nonionic polymer hydroxypropyl methylcellulose was prepared according to Table 3.

The water phase and the oil phase were each heated separately with mixing to about 170-180° F. The hydroxypropyl methylcellulose was added to the water phase at about 170° F. The oil phase was then added to the water phase with adequate stirring. When thoroughly mixed and homogenized, the emulsion was cooled to 120-125° F. and the other ingredients were added. These were thoroughly mixed and cooled to about 105° F. prior to filling.

TABLE 3
SKIN CARE COMPOSITION CONTAINING SALICYLIC ACID, TWO
EMULSIFIERS, HIGH PROPORTION OF PROPYLENE GLYCOL.
AND HYDROXYPROPYL METHYLCELLULOSE

| | CONCENTRATION | PREFERRED |
|---|---|---|
| INGREDIENT | RANGE. % | CONCENTRATION % |
| Water Phase | | |
| Water | 40.0-70.0 | 52.94 |
| Propylene Glycol | 15.0-30.0 | 20.00 |
| Tetrasodium EDTA | 0.05-0.20 | 0.15 |
| Ceteareth-25 | 1.50-4.00 | 3.20 |
| Hydroxypropyl | 0.05-0.30 | 0.16 |
| Methylcellulose | | |
| Oil Phase | | |
| Lanolin Oil | 3.00-10.00 | 7.00 |
| Caprylic/Capric | 1.00-4.00 | 2.00 |
| Triglycerides | | |
| Dimethicone | 0.50-3.00 | 1.40 |
| Cetyl Alcohol | 3.00-7.00 | 3.10 |
| Stearyl Alcohol | 2.00-5.00 | 2.05 |
| Ceteareth-6 with | 2.00-4.00 | 3.00 |
| Stearyl Alcohol | | |
| Glyceryl Stearate | 1.00-4.00 | 2.20 |
| Salicylic Acid | 1.80-3.00 | 2.00 |
| Butylated | 0.05-2.00 | 0.10 |
| Hydroxytoluene | | |
| Vitamin A and | 0.005-0.50 | 0.01 |
| Vitamin D₃ | | |
| Other Ingredients | | |
| Methylparaben | 0.10-0.50 | 0.25 |
| Propylparaben | 0.005-0.40 | 0.20 |
| 2-Methyl-4- | 0.01-0.06 | 0.04 |
| isothiazolin-3-one | | |
| Fragrance | 0.10-0.50 | 0.20 |

A cream prepared according to Table 3 did not exhibit any salicylic acid shard formation over time and formed a stable emulsion that is capable of surviving accelerated aging at 40° C. for six months.

### Example 4

### Skin Care Composition Containing Salicylic Acid, Two Emulsifiers, High Proportion of Propylene Glycol and Polyquaternium-10

A skin care composition according to the present invention containing salicylic acid, two emulsifiers, a high proportion of propylene glycol, and the cationic polymer polyquaternium-10 was prepared according to Table 4. The composition was prepared according to the method used for Example 3; the polyquaternium-10 was in the water phase. The skin care composition of Example 4 did not exhibit any salicylic acid shard formation overtime and formed a stable emulsion that is capable of surviving accelerated aging at 40° C. for six months.

TABLE 4
SKIN CARE COMPOSITION CONTAINING SALICYLIC ACID, TWO
EMULSIFIERS HIGH PROPORTION OF PROPYLENE GLYCOL.
AND POLYQUATERNIUM-10

| | CONCENTRATION | PREFERRED |
|---|---|---|
| INGREDIENT | RANGE. % | CONCENTRATION % |
| Water Phase | | |
| Water | 40.0-70.0 | 52.49 |
| Propylene Glycol | 15.0-30.0 | 20.00 |
| Tetrasodium EDTA | 0.05-0.20 | 0.15 |
| Ceteareth-25 | 1.50-3.50 | 3.20 |
| Polyquaternium-10 | 0.05-0.30 | 0.16 |
| Oil Phase | | |
| Lanolin Oil | 3.0-10.0 | 7.00 |
| Caprylic/Capric | 1.0-4.0 | 2.00 |
| Triglycerides | | |
| Dimethicone | 0.5-3.0 | 1.40 |
| Cetyl Alcohol | 3.0-7.0 | 3.30 |
| Stearyl Alcohol | 2.0-5.0 | 2.30 |
| Ceteareth-6 with | 2.0-5.0 | 3.00 |
| Stearyl Alcohol | | |
| Glyceryl Stearate | 1.0-3.0 | 2.20 |
| Salicylic Acid | 1.8-3.0 | 2.00 |
| Butylated | 0.05-0.9 | 0.10 |
| Hydroxytoluene | | |
| Vitamin A and | 0.005-0.50 | 0.01 |
| Vitamin D₃ | | |
| Other Ingredients | | |
| Methylparaben | 0.10-0.50 | 0.25 |
| Propylparaben | 0.10-0.50 | 0.20 |
| 2-Methyl-4- | 0.01-0.06 | 0.04 |
| isothiazolin-3-one | | |
| Fragrance | 0.10-0.50 | 0.20 |

### Example 5

### Skin Care Composition Containing Salicylic Acid, Two Emulsifiers, Propylene Glycol, Ethoxydiglycol, and Hydroxypropyl Methylcellulose

A skin care composition according to the present invention containing salicylic acid, two emulsifiers, propylene glycol, ethoxydiglycol, and hydroxypropyl methylcellulose was prepared according to Table 5. The composition was prepared according to the method used for Example 3. The hydroxypropyl methylcellulose was added to the water phase at about 170° F. The ethoxydiglycol was added to the oil phase.

TABLE 5
SKIN CARE COMPOSITION CONTAINING SALICYLIC ACID TWO
EMULSIFIERS PROPYLENE GLYCOL ETHOXYDIGLYCOL AND
HYDROXYPROPYL METHYLCELLULOSE

| | CONCENTRATION | PREFERRED |
|---|---|---|
| INGREDIENT | RANGE, % | CONCENTRATION % |
| Water Phase | | |
| Water | 40.0-70.0 | 53.89 |
| Propylene Glycol | 5.0-20.0 | 15.00 |
| Tetrasodium EDTA | 0.05-2.00 | 0.15 |
| Ceteareth-25 | 1.50-4.00 | 3.20 |
| Hydroxypropyl | 0.05-0.30 | 0.16 |
| Methylcellulose | | |
| Oil Phase | | |
| Lanolin Oil | 3.00-10.00 | 7.00 |
| Ethoxydiglycol | 1.00-6.00 | 3.00 |
| Caprylic/Capric | 1.00-4.00 | 2.00 |
| Triglycerides | | |
| Dimethicone | 0.50-3.00 | 1.40 |
| Cetyl Alcohol | 3.00-7.00 | 3.60 |
| Stearyl Alcohol | 2.00-5.00 | 2.60 |
| Ceteareth-6 with | 2.00-5.00 | 3.00 |
| Stearyl Alcohol | | |
| Glyceryl Stearate | 1.00-4.00 | 2.20 |
| Salicylic Acid | 1.80-3.00 | 2.00 |
| Butylated | 0.05-0.90 | 0.10 |
| Hydroxytoluene | | |
| Vitamin A and | 0.005-0.50 | 0.01 |
| Vitamin D₃ | | |
| Other Ingredients | | |
| Methylparaben | 0.10-0.50 | 0.25 |
| Propylparaben | 0.10-0.50 | 0.20 |
| 2-Methyl-4- | 0.01-0.06 | 0.04 |
| isothiazolin-3-one | | |
| Fragrance | 0.10-0.50 | 0.20 |

The skin care composition of Example 5 did not exhibit any salicylic acid shard formation over time and formed a stable emulsion that is capable of surviving accelerated aging at 40° C. for six months.

### Example 6

### Occurrence of Salicylic Acid Shards in Creams and Ointments

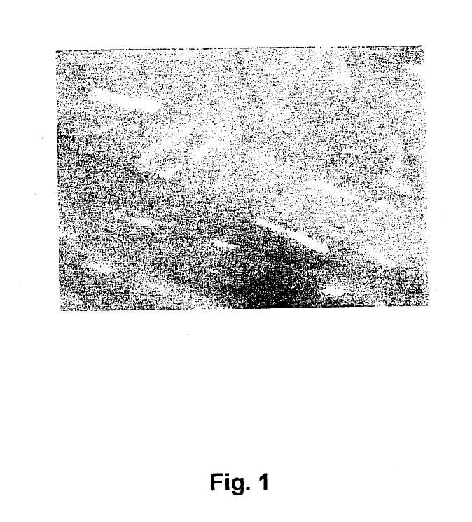

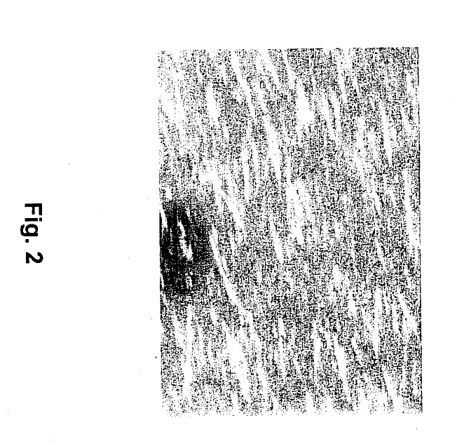

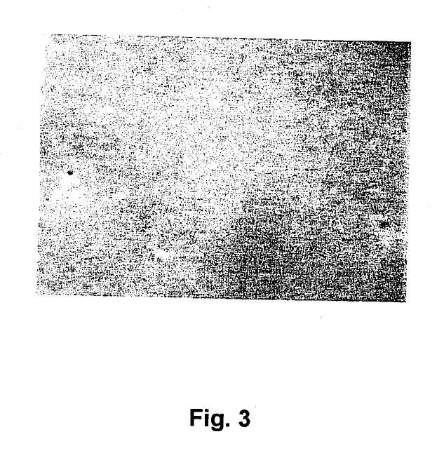

FIGS. 1-3 are photomicrographs showing the occurrence or non-occurrence of salicylic acid shards in creams and ointments. FIG. 1 shows the occurrence of salicylic acid crystals (shards) after 5 weeks of aging in cream containing 7% propylene glycol. FIG. 2 shows the occurrence of salicylic acid shards in a commercial ointment known as “Skin Plaque.” FIG. 3 shows the absence of salicylic acid shards after 4 months of aging in a cream containing 20% of propylene glycol.

The present invention provides oil-in-water emulsions containing salicylic acid and propylene glycol, together with other ingredients. The emulsions are of improved stability. These emulsions are stable over long periods when stored at room temperature, obviating the need for refrigeration. These emulsions are suitable for treatment of psoriasis and other skin conditions.

Although the present invention has been described in considerable detail, with reference to certain preferred versions thereof, other versions and embodiments are possible. Therefore, the scope of the invention is determined by the following claims.

While the specification describes particular embodiments of the present invention, those of ordinary skill can devise variations of the present invention without departing from the inventive concept.

We claim:

1. A skin care composition comprising an oil in-water emulsion, comprising:
(a) water;
(b) salicylic acid in an effective quantity;
(c) lanolin oil in an effective quantity;
(d) a complex comprising Vitamin A and Vitamin D₃ in an effective quantity;
(e) an emulsifier component in a quantity sufficient to effectively emulsify oil and water phases, the emulsifier component comprising a nonionic ether surfactant with a HLB of about 12 to about 18; and
(f) a solvent component in an effective quantity, wherein the pH of the composition is from about 2.0 to about 4.0.

2. The skin care composition of claim 1, wherein the nonionic ether surfactant is selected from the group consisting of ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-19, ceteareth-20, ceteareth-21, ceteareth-22, ceteareth-23, ceteareth-24, ceteareth-25, ceteareth-26, ceteareth-27, ceteareth-28, ceteareth-29 and ceteareth-30.

3. The skin care composition of claim 1, wherein the composition further comprises dimethicone.

4. The skin care composition of claim 1, wherein the composition further comprises at least one of a plant extract and a herbal extract.

5. The skin care composition of claim 1, wherein the solvent component comprises at least one solvent selected from the group consisting of propylene glycol, ethylene glycol, 1,3-butylene glycol, sorbitol, and glycerol.

6. The skin care composition of claim 1, wherein the composition further comprises a long-chain fatty acid ester of glycerol selected from the group consisting of glyceryl stearate, glyceryl palmitate, and glyceryl arachidate.

7. The skin care composition of claim 1, wherein the composition further comprises a lipid-soluble long-chain alcohol component.

8. The skin care composition of claim 7, wherein the lipid-soluble long-chain alcohol component comprises at least one long-chain lipid-soluble alcohol selected from the group consisting of stearyl alcohol and cetyl alcohol.

9. The skin care composition of claim 1, wherein the composition further comprises caprylic/capric triglycerides.

10. The skin care composition of claim 1, wherein the composition further comprises a preservative component.

11. The composition of claim 10, wherein the preservative component further comprises at least one preservative selected from the group consisting of propylparaben, methylparaben, and 2-methyl-4-isothiazolin-3-one.

12. The skin care composition of claim 1, wherein the composition further comprises an antioxidant component.

13. The skin care composition of claim 12, wherein the antioxidant component comprises at least one antioxidant selected from the group consisting of butylated hydroxytoluene, butylated hydroxyanisole, and a complex comprising propyl gallate, propylene glycol, and citric acid.

14. The skin care composition of claim 1, wherein the composition further comprises a chelator component.

15. The skin care composition of claim 14, wherein the chelator component is tetrasodium ethyl enediaminetetraacetic acid.

16. A skin care composition comprising an oil in-water emulsion comprising:
(a) from about 40.0% to about 80.0% of water;
(b) from about 1.8% to about 3.0% of salicylic acid;
(c) from about 2.0% to about 20.0% of lanolin oil;
(d) from about 0.005% to about 0.50% of a complex comprising Vitamin A and Vitamin D₃;
(e) from about 1.0% to about 6.0% of ceteareth-25;
(f) from about 2.0% to about 35.0% of propylene glycol;
(g) from about 0.05% to about 0.50% of tetrasodium ethylenediaminetetraacetic acid;
(h) from about 0.5% to about 5.0% of caprylic/capric triglycerides;
(i) from about 0.5% to about 5.0% of dimethicone;
(j) from about 2.0% to about 12.0% of cetyl alcohol;
(k) from about 1.0% to about 10.0% stearyl alcohol;
(l) from about 1.0% to about 5.0% of glyceryl stearate;
(m) from about 0.1% to about 1.0% of butylated hydroxytoluene;
(n) from about 0.10% to about 0.50% of propylparaben;
(o) from about 0.10% to about 0.50% of methylparaben;
(p) from about 0.005% to about 0.08% of 2-methyl-4-isothiazolin-3-one;
(q) from about 0.10% to about 0.40% of fragrance;
(r) optionally, from about 0.025% to about 2.5% of at least one plant or herbal extract; and
(s) optionally, from about 0.01% to about 1.5% of allantoin.

17. A method for treating a dermatological or systemic inflammatory condition, comprising the step of applying the skin care composition of claim 1 to the skin of a patient in a therapeutically effective quantity.

18. The method of claim 17, wherein the dermatological or systemic inflammatory condition is selected from the group consisting of psoriasis, seborrheic dermatitis and dandruff.

19. The method of claim 17, further comprising the step of administering at least one additional topically or systemically administered agent for the treatment of psoriasis.

20. A skin care composition comprising an oil-in-water emulsion comprising:
(a) water;
(b) a hydroxy acid in an effective quantity, the hydroxy acid being selected from the group consisting of α-hydroxy acids, β-hydroxy acids, and multi-hydroxy acids;
(c) lanolin oil in an effective quantity;
(d) a complex comprising Vitamin A and Vitamin D₃ in an effective quantity;
(e) an emulsifier component in a quantity sufficient to effectively emulsify oil and water phases, the emulsifier component comprising a nonionic ether surfactant with a HLB of about 12 to about 18;
(f) a solvent component in an effective quantity; and
(g) optionally, allantoin in an effective quantity, wherein the pH of the composition is from about 2.0 to about 4.0.

21. A method for treating a dermatological or systemic inflammatory condition, comprising the step of applying the skin care composition of claim 20 to the skin of a patient in a therapeutically effective quantity.

22. The method of claim 21, wherein the dermatological or systemic inflammatory condition is selected from the group consisting of psoriasis, seborrheic dermatitis and dandruff.

23. The method of claim 21, further comprising the step of administering at least one additional topically or systemically administered agent for the treatment of psoriasis.

24. A skin care composition comprising an oil-in-water emulsion, comprising:
(a) water;
(b) salicylic acid in an effective quantity;
(c) lanolin oil in an effective quantity;
(d) a complex comprising Vitamin A and Vitamin D₃ in an effective quantity;
(e) an emulsifier component in a quantity sufficient to effectively emulsify oil and water phases, the emulsifier component comprising:
(i) a first nonionic polyoxyethylene ether with a HLB of about 15 to about 17; and
(ii) a second nonionic polyoxyethylene ether with a HLB of about 10 to about 12;
(f) at least one glycol in a quantity sufficient to prevent formation of salicylic acid crystals; and
(g) optionally, allantoin in an effective quantity.

25. The skin care composition of claim 24, wherein the at least one glycol is selected from the group consisting of propylene glycol, ethoxydiglycol, and a mixture thereof.

26. The skin care composition of claim 24, wherein the first nonionic polyoxyethylene ether is ceteareth-25.

27. The skin care composition of claim 24, wherein the second nonionic polyoxyethylene ether is ceteareth-6 with stearyl alcohol.

28. The skin care composition of claim 24, wherein the composition further comprises dimethicone.

29. The skin care composition of claim 24, wherein the composition further comprises at least one of a plant extract and a herbal extract.

30. The skin care composition of claim 24, wherein the composition further comprises a long-chain fatty acid ester of glycerol selected from the group consisting of glyceryl stearate, glyceryl palmitate, and glyceryl arachidate.

31. The skin care composition of claim 24, wherein the composition further comprises caprylic/capric triglycerides.

32. The skin care composition of claim 24, wherein the composition further comprises a preservative component.

33. The composition of claim 32, wherein the preservative component further comprises at least one preservative selected from the group consisting of propylparaben, methylparaben, and 2-methyl-4-isothiazolin-3-one.

34. The skin care composition of claim 24, wherein the composition further comprises an antioxidant component.

35. The skin care composition of claim 34, wherein the antioxidant component comprises at least one antioxidant selected from the group consisting of butylated hydroxytoluene, butylated hydroxyanisole, and a complex comprising propyl gallate, propylene glycol, and citric acid.

36. The skin care composition of claim 24, wherein the composition further comprises a chelator component.

37. The skin care composition of claim 36, wherein the chelator component is tetrasodium ethylenediaminetetraacetic acid.

38. The skin care composition of claim 24, wherein the composition further comprises a lipid-soluble long-chain alcohol component.

39. The skin care composition of claim 38, wherein the lipid-soluble long-chain alcohol component comprises at least one lipid-soluble long-chain alcohol selected from the group consisting of stearyl alcohol and cetyl alcohol.

40. The skin care composition of claim 24, wherein the composition further comprises a hydrophilic carbohydrate-containing polymer in an effective quantity.

41. The skin care composition of claim 40, wherein the hydrophilic carbohydrate-containing polymer is selected from the group consisting of hydroxypropyl methylcellulose, hydroxyethyl ethylcellulose, methylcellulose, ethylcellulose, acacia gum, tragacanth gum, locust bean gum, guar gum, gum arabic, xanthan gum, and agarose.

42. The skin care composition of claim 24, wherein the composition further comprises a quaternary ammonium-containing polymer in an effective quantity.

43. The skin care composition of claim 42, wherein the quaternary ammonium-containing polymer is polyquaternium-10.

44. A skin care composition comprising an oil-in-water emulsion, comprising:
(a) from about 40.0% to about 70.0% of water;
(b) from about 15.0% to about 25.0% of propylene glycol;
(c) from about 0.05% to about 0.20% of tetrasodium ethylenediaminetetraacetic acid;
(d) from about 1.50% to about 4.00% of ceteareth-25;
(e) from about 3.00% to about 10.00% of lanolin oil;
(f) from about 1.00% to about 4.00% of caprylic/capric triglycerides;
(g) from about 0.50% to about 3.00% of dimethicone;
(h) from about 3.00% to about 7.00% of cetyl alcohol;
(i) from about 2.00% to about 5.00% of stearyl alcohol;
(j) from about 1.00% to about 3.00% of glyceryl stearate;
(k) from about 2.00% to about 4.00% of ceteareth-6 with stearyl alcohol;
(l) from about 1.80% to about 3.00% of salicylic acid;
(m) from about 0.05% to about 2.0% of butylated hydroxytoluene;
(n) from about 0.005% to about 0.50% of a complex comprising Vitamin A and Vitamin D₃;
(o) from about 0.10% to about 0.50% of methylparaben;
(p) from about 0.05% to about 0.50% of propylparaben;
(q) from about 0.01 % to about 0.08% of 2-methyl-4-isothiazolin-3-one;
(r) from about 0.10% to about 0.50% of fragrance;
(s) optionally, from about 0.025% to about 0.80% of at least one of a plant and a herbal extract; and
(t) optionally, from about 0.01% to about 1.5% of allantoin.

45. A method for treating a dermatological or systemic inflammatory condition, comprising the step of applying the skin care composition of claim 44 to the skin of a patient in a therapeutically effective quantity.

46. The method of claim 45, wherein the dermatological or systemic inflammatory condition is selected from the group consisting of psoriasis, seborrheic dermatitis and dandruff.

47. The method of claim 45, further comprising the step of administering at least one additional topically or systemically administered agent for the treatment of psoriasis.

48. A skin care composition comprising an oil-in-water emulsion comprising:
(a) water;
(b) a hydroxy acid in an effective quantity, the hydroxy acid being selected from the group consisting of α-hydroxy acids, β-hydroxy acids, and multi-hydroxy acids;
(c) lanolin oil in an effective quantity;
(d) a complex comprising Vitamin A and Vitamin D₃ in an effective quantity;
(e) an emulsifier component in a quantity sufficient to effectively emulsify oil and water phases, the emulsifier component comprising:
(i) a first nonionic polyoxyethylene ether with a HLB of about 15 to about 17; and
(ii) a second nonionic polyoxyethylene ether with a HLB of about 10 to about 12;
(f) a glycol in a quantity sufficient to prevent formation of crystals of the hydroxy acid; and
(g) optionally, allantoin in an effective quantity.

49. The skin care composition of claim 48, wherein the glycol is selected from the group consisting of propylene glycol, ethoxydiglycol, and a mixture thereof.

50. The skin care composition of claim 48, wherein the composition further comprises a hydrophilic carbohydrate-containing polymer in an effective quantity.

51. The skin care composition of claim 50, wherein the hydrophilic carbohydrate containing polymer is selected from the group consisting of hydroxypropyl methylcellulose, hydroxyethyl ethylcellulose, methylcellulose, ethylcellulose, acacia gum, tragacanth gum, locust bean gum, guar gum, gum arabic, xanthan gum, and agarose.

52. The skin care composition of claim 48, wherein the composition further comprises a quaternary ammonium-containing polymer in an effective quantity.

53. The skin care composition of claim 52, wherein the quaternary ammonium-containing polymer is polyquaternium-10.

54. A method for treating a dermatological or systemic inflammatory condition comprising the step of applying the skin care composition of claim 48 to the skin of a patient in a therapeutically effective quantity.

55. The method of claim 54, wherein the dermatological or systemic inflammatory condition is selected from the group consisting of psoriasis, seborrheic dermatitis and dandruff.

56. The method of claim 54, further comprising the step of administering at least one additional topically or systemically administered agent for the treatment of psoriasis.

57. A skin care composition comprising an oil-in-water emulsion, comprising:
(a) from about 40.0% to about 70.0% of water;
(b) from about 15.0% to about 30.0% of propylene glycol;
(c) from about 0.05% to about 0.20% of tetrasodium ethylenediaminetetraacetic acid;
(d) from about 1.50% to about 4.00% of ceteareth-25;
(e) from about 0.05% to about 0.30% of hydroxypropyl methylcellulose;
(f) from about 3.00% to about 10.0% of lanolin oil;
(g) from about 1.00% to about 4.00% of caprylic/capric triglycerides;
(h) from about 0.50% to about 3.00% of dimethicone;
(i) from about 3.00% to about 7.00% of cetyl alcohol;
(j) from about 2.00% to about 5.00% of stearyl alcohol;
(k) from about 2.00% to about 5.00% of ceteareth-6 with stearyl alcohol;
(l) from about 1.00% to about 4.00% of glyceryl stearate;
(m) from about 1.80% to about 3.00% of salicylic acid;
(n) from about 0.05% to about 2.00% of butylated hydroxytoluene;
(o) from about 0.005% to about 0.50% of a complex comprising Vitamin A and Vitamin D3;
(p) from about 0.10% to about 0.50% of methylparaben;
(q) from about 0.05% to about 0.50% of propylparaben;
(r) from about 0.01% to about 0.06% of 2-methyl-4-isothiazolin-3-one;
(s) from about 0.10% to about 0.50% of fragrance;
(t) optionally, from about 0.025% to about 0.50% of at least one plant or herbal extract; and
(u) optionally, from about 0.01% to about 1.5% of allantoin.

58. The skin care composition of claim 57, wherein the composition further comprises from about 0.05% to about 0.30% of polyquaternium-10.

59. A method for treating a dermatological or systemic inflammatory condition, comprising the step of applying the skin care composition of claim 57 to the skin of a patient in a therapeutically effective quantity.

60. The method of claim 59, wherein the dermatological or systemic inflammatory condition is selected from the group consisting of psoriasis, seborrheic dermatitis and dandruff.

61. The method of claim 59, further comprising the step of administering at least one additional topically or systemically administered agent for the treatment of psoriasis.

62. A skin care composition, comprising:
(a) a shell comprising agar or gelatin;
(b) a solution of salicylic acid dissolved in at least one glycol, the solution being encapsulated in the shell; and
(c) a carrier for the shell, the carrier being in the form of a cream, gel, or ointment, the shell being broken to release the solution of salicylic acid when the composition is applied to the skin of a user.

63. The skin care composition of claim 62, wherein the shell is agar.

64. The skin care composition of claim 62, wherein the shell is gelatin.

65. The skin care composition of claim 62, wherein the carrier is in the form of a cream.

66. The skin care composition of claim 65, wherein the cream is an oil-in-water emulsion.

67. The skin care composition of claim 66, wherein the oil-in-water emulsion further comprises a complex comprising Vitamin A and Vitamin D₃ and lanolin oil.

68. The skin care composition of claim 62, wherein the carrier is in the form of an ointment.

69. The skin care composition of claim 62, wherein the carrier is in the form of a gel.

70. The skin care composition of claim 62, wherein the carrier comprises about 70% by weight of the composition and the shell and encapsulated solution of salicylic acid comprise about 30% by weight of the composition.

71. The skin care composition of claim 65 wherein the glycol is selected from the group consisting of propylene glycol, ethoxydiglycol, and a mixture thereof.
